(19) United States
(12) Patent Application Publication
(10) Pub. No.: US 2026/0311798 A1
(43) Pub. Date: Oct. 8, 2026

(54) GUIDANCE FRAMEWORK FOR GENEALOGICAL-RESEARCH JOURNEYS

(71) Applicant: Ancestry.com Operations Inc., Lehi, UT (US)

(72) Inventors: Madhu Shivashankaraiah, Redmond, WA (US); Kevin Niels Christiansen, Lindon, UT (US); Ronald Glen Greenwood, Highland, UT (US); Karl Walter Judson, Cedar Hills, UT (US)

(21) Appl. No.: 19/636,465

(22) Filed: Apr. 1, 2026

Related U.S. Application Data
(60) Provisional application No. 63/783,849, filed on Apr. 4, 2025.

(51) Int. Cl. G16B 10/00; G16B 45/00

(52) U.S. Cl. CPC G16B 10/00; G16B 45/00

(57) ABSTRACT

The present disclosure relates to methods, systems, and non-transitory computer-readable media for providing a user account with personalized guidance along a genealogical-research journey to achieve a genealogical-research target (or goal). For instance, the disclosed systems can leverage user-account data to determine a genealogical-research target and can generate and/or adaptively augment a genealogical-research journey to achieve the target. In some embodiments, the genealogical-research journey includes guidance in the form of a set of discrete functions and a journey-progress indicator that defines which discrete functions are (or have been) executed along the genealogical-research journey. Based on the journey-progress indicator and from among the set of discrete functions, the disclosed systems can determine an advancing function that, upon execution, progresses the genealogical-research journey further toward the genealogical-research target. In some cases, the disclosed systems provide a notification of the advancing function for display on a client device associated with the user account.

### CROSS-REFERENCE TO RELATED APPLICATIONS

This application claims priority to and the benefit of U.S. Provisional Patent Application No. 63/783,849, filed on Apr. 4, 2025, which is incorporated herein by reference in its entirety.

### BACKGROUND

In the field of genealogical research, existing systems can provide certain genealogical research functions for navigating among available records, generating genealogy trees, and performing text-based searches. Using existing tools, some systems can identify genealogical records, determine relationships among individuals, and/or identify additional genealogical data to associate or correlate with an individual. However, existing systems exhibit a number of deficiencies or drawbacks, particularly in relation to adaptability and efficiency.

As just suggested, existing systems often provide a generalized, one-size-fits-all set of online genealogical research tools and resources, such as genealogical records, that are not adaptive to specific research tasks or goals. For instance, the tools and resources in most existing systems require expertise from users to know how to implement the tools, where to access the tools, and on what data the tools are effective. Further, given the massive volume of extant genealogical records associated with existing genealogical-research services, identifying via, e.g., a person-specific query, a pertinent record, can be akin to finding a particular needle in a bunch of stacks that comprise billions of needles, requiring impractical—even impossible—amounts of time and effort on a user’s part to parse page after page after page of potentially relevant records for a particular person. Indeed, existing systems provide an unstructured set of tools and resources accessible independently in their interfaces and/or applications which sometimes require significant experience (and processing expense) to adapt to one another in a pipeline when performing genealogical research to determine a particular piece of information or access a particular genealogical record.

For example, a new user of a genealogical-research service, desiring to learn more about stories of their ancestor, such as how their ancestors migrated, what their ancestors looked like, whether and in what way their ancestors participated in historically significant events such as wars, and other emotionally meaningful stories, is faced with the daunting process of first building a family tree to identify who their ancestors are, determine which of those ancestors are likely to have such stories, piece together genealogical and historical records that tell pieces of those stories, and so on.

Such tasks can require the successful utilization by the user of a variety of tools and associated resources offered by a genealogical-research service, such as a genetic-match determination tool, one or more of an ethnicities, communities, and traits-prediction tool, a family tree-building tool, a record-search tool, a hints-generation tool, and so on. All of these tasks can be overwhelming for new users who are uninitiated in the field of genealogical research and who may not even be aware of the existence of such tools, the necessity of such tools to achieving their particular genealogical-research goal, and the proper use of such tools to parse a large volume of historical records, user-generated content, genetic matches and groups, publicly available genealogy trees, and other resources.

Due at least in part to their inflexible natures, existing systems are also inefficient. Specifically, the unstructured and disjointed (and sometimes incompatible) nature of available tools in existing systems often requires excessive time and processing power (in addition to onerous user interactions) to implement multiple tools together as part of a single genealogical-research journey (e.g., for a common goal of determining a piece of information or identifying a record). For example, existing systems often require various data reformatting steps to improve compatibility among tools, in addition to onerous navigational inputs to access and apply tools housed in their own respective interfaces or applications. Consequently, the disjointed structure of existing systems results in excessive use of computational resources, such as processing power and memory, to identify and use multiple models together in a single pipeline. Additionally, existing systems require excessive navigational inputs via a client device through various databases, records, and/or tools to determine a desired piece of genealogical information, access a target genealogical record, or achieve some other genealogical-research target (or goal).

These along with additional problems and issues exist with regard to existing systems.

### SUMMARY

This disclosure describes one or more embodiments of systems, methods, and non-transitory computer-readable storage media that provide benefits and/or solve one or more of the foregoing and other problems in the art. In particular, the disclosed systems introduce a unique, unified approach to providing a user account with personalized guidance along or during a genealogical-research journey to achieve a genealogical-research target (or goal). For instance, the disclosed systems utilize a unified data architecture to analyze user-account data associated with a user account within a genealogical-data system and personalize the genealogical-research journey through intelligent and predictive instruction generation (and/or execution). As an example, the disclosed systems can leverage user-account data to determine a genealogical-research target and can generate and/or adaptively augment a genealogical-research journey that includes one or more models, processes, and/or subroutines which, when executed, achieve the target.

In some embodiments, the genealogical-research journey includes guidance in the form of a set of discrete functions and a journey-progress indicator that defines which discrete functions are (or have been) executed along or dring the genealogical-research journey. Based on the journey-progress indicator and from among the set of discrete functions, the disclosed systems can determine an advancing function that, upon execution, progresses the genealogical-research journey further toward the genealogical-research target. In some cases, the disclosed systems provide a notification of the advancing function for display on a client device associated with the user account. In embodiments, the disclosed systems modify a node and/or an edge of a cluster database of a genealogical database in response to a user action and/or in response to a determined advancing function as part of the genealogical-research journey.

### BRIEF DESCRIPTION OF THE DRAWINGS

The detailed description provides one or more embodiments with additional specificity and detail through the use of the accompanying drawings, as briefly described below.

FIG. 1 illustrates a schematic diagram of an example system environment for implementing a genealogical-guidance system in accordance with one or more embodiments.

FIG. 2 illustrates an example diagram of determining an advancing function for a genealogical-research journey and providing a notification of the advancing function to a client device in accordance with one or more embodiments.

FIG. 3 illustrates an example diagram of analyzing user-account data to personalize a genealogical-research journey for a user account in accordance with one or more embodiments.

FIG. 4 illustrates an example diagram of generating a genealogical-research journey to achieve a genealogical-research target in accordance with one or more embodiments.

FIG. 5 illustrates an example diagram of utilizing guidance scaffolding to determine an advancing function in accordance with one or more embodiments.

FIG. 6 illustrates an example timeline of various acts performed in a first genealogical-research journey and in a second genealogical-research journey in accordance with one or more embodiments.

FIGS. 7A-7E illustrate example user interfaces of a client device for determining a genealogical-research target and executing various advancing functions in accordance with one or more embodiments.

FIG. 8 illustrates an example diagram of a genealogical-data system interfacing with a genealogical database in accordance with one or more embodiments.

FIG. 9 illustrates an example flowchart of a series of acts for providing a notification of an advancing function in accordance with one or more embodiments.

FIG. 10 illustrates a block diagram of an exemplary computing device for implementing one or more embodiments of the present disclosure.

FIG. 11 illustrates an example environment of a genealogical-data system including a genealogical-guidance system in accordance with one or more embodiments.

### DETAILED DESCRIPTION

This disclosure describes one or more embodiments of a genealogical-guidance system 100 that can provide a user account with personalized guidance along a personalized genealogical-research journey to achieve a genealogical-research target (or goal). For instance, the genealogical-guidance system 100 receives or determines an indication of a genealogical-research target (e.g., in response to a text query provided via an agent interface, a user selection of a genealogical-research target from a plurality of predetermined genealogical-research targets presented in a user interface of a client device, and/or a prediction generated based on user account behavior) and analyzes user-account data associated with a user account within a genealogical-data system to personalize a genealogical-research journey to achieve the genealogical-research target. As an example, the disclosed systems can leverage the user-account data to determine the genealogical-research target for the user account and to generate and/or adaptively augment the genealogical-research journey for the user account based on the genealogical-research target. In some embodiments, the genealogical-research journey includes personalized guidance in the form of a set of discrete functions that can provide hints, assistance, suggestions, recommendations, direction, and/or automated execution of functions for the user account along or during the genealogical-research journey and based on the genealogical-research target.

To elaborate, the genealogical-guidance system 100 can determine a journey-progress indicator that can define which discrete functions are (or have been) executed along or dring the genealogical-research journey. Based on the journey-progress indicator and from among the set of discrete functions, the genealogical-guidance system 100 can determine an advancing function that, upon execution, progresses the genealogical-research journey toward the genealogical-research target. In at least one embodiment, the genealogical-guidance system 100 provides a notification of the advancing function for display on a client device associated with the user account. In some cases, the genealogical-guidance system 100 executes the advancing function to advance the genealogical-research journey.

Embodiments of the genealogical-guidance system 100 can provide improvements or advantages over existing systems. For instance, the genealogical-guidance system 100 can improve adaptability relative to existing systems. As an example, rather than providing a generalized set of online genealogical research tools and resources that are not adaptive to specific research tasks or goals or to individual user accounts, the genealogical-guidance system 100 can provide a user account with personalized guidance along or during a genealogical-research journey towards a genealogical-research target. To elaborate, the genealogical-guidance system 100 can analyze (and collect) user-account data associated with a user account and can leverage the user-account data to generate a personalized genealogical-research journey for the user account. Indeed, the genealogical-guidance system 100 can leverage the user-account data to personalize various aspects of the genealogical-research journey, including determining a genealogical-research target for the genealogical-research journey, determining a set of discrete functions for the genealogical-research journey, and/or determining an advancing function from among the set of discrete functions which will, upon execution, progress the genealogical-research journey toward the genealogical-research target. The genealogical-guidance system 100 can, therefore, personalize the genealogical-research journey to the needs and desires of the user account.

The genealogical-guidance system 100 can further improve adaptability by utilizing a unique form of guidance scaffolding to adapt a personalized genealogical-research journey to the research needs and/or research targets (or goals) of a user account. In particular, the guidance scaffolding can improve adaptability using one or more journey-progress indicators and/or an orchestrator such as a journey orchestration model. For example, by utilizing the one or more journey-progress indicators, the genealogical-guidance system 100 is capable of concurrently generating, tracking, categorizing, storing, and/or restoring (or resuming) data associated with one or more genealogical-research journeys associated with a single user account. Indeed, the genealogical-guidance system 100 can not only utilize the one or more journey-progress indicators to define which discrete functions are (or have been) executed along or during a single genealogical-research journey but also which are (or have been) executed along or during a plurality of genealogical-research journeys. To elaborate, the genealogical-guidance system 100 can utilize a journey-progress indicator to categorize and store data associated with discrete functions that are (or have been) executed along or during a plurality of genealogical-research journeys, e.g., for a plurality of genealogical-research journeys associated with a user account. By leveraging these data, the genealogical-guidance system 100 can continue a particular genealogical-research journey at the point (e.g., at a particular function in the set of discrete functions) in the genealogical-research journey where it was paused and/or otherwise discontinued (e.g., when another genealogical-research journey was initiated or continued).

As mentioned above, the guidance scaffolding (e.g., which is utilized by, and/or included within, the genealogical-guidance system 100) can improve adaptability using the orchestrator or journey orchestration model (e.g., an artificial intelligence or AI model). In particular, the journey orchestration model can agentically access and/or utilize one or more skill sets to resolve particular issues according to the genealogical-research needs of a user account. For instance, based on user-account data, the one or more journey-progress indicators, and/or one or more sets of discrete functions, the genealogical-guidance system 100 can use the journey orchestration model to agentically determine and/or select which skill set from a plurality of candidate skill sets may be (e.g., is predicted to be) well-suited for determining and/or executing an advancing function for a particular genealogical-research journey. By intelligently orchestrating the plurality of candidate skill sets in this way, the genealogical-guidance system 100 can dynamically and flexibly adapt to individual user-account needs and/or genealogical-research contexts. This dynamism and flexibility is of particular importance in the space of genealogical research, where every user account—representing individuals researching their family history—is associated with a unique combination of ancestors, relatives, and genealogical-research goals and resources associated therewith.

In addition to improving functionality, the genealogical-guidance system 100 also improves efficiency relative to existing systems. For instance, in contrast with the disjointed structure of existing systems that result in excessive use of computational resources (such as processing power and memory) when trying to use multiple models together in a single pipeline, the genealogical-guidance system 100 more seamlessly implements models using its guidance scaffolding. To elaborate, the guidance scaffolding can define a collaborative structure of otherwise disparate databases and models that support and/or orchestrate a genealogical-research journey. In some cases, the guidance scaffolding efficiently drives the progression of genealogical-research tasks (e.g., to determine an advancing function) and/or provides structured assistance, decision-making processes, and/or automation capabilities. Additionally, by utilizing the guidance scaffolding in combination with user-account data, the genealogical-guidance system 100 can guide a user along the genealogical-research journey without requiring excessive time or navigational inputs via a client device painstakingly wading through various databases, records, and/or tools to achieve (or progress towards) a genealogical-research target (if indeed the user continues to progress towards their genealogical-research target rather than becoming discouraged and giving up).

As illustrated by the foregoing (and following) discussion, the present disclosure utilizes a variety of terms to describe features and advantages of the genealogical-guidance system 100. Additional detail is now provided regarding the meaning of such terms. As used herein, the term “genealogical-research journey” refers to a structured, personalized set or sequence of functions, processes, or subroutines that, when executed, achieve or at least make progress in pursuit of a genealogical-research target. In particular, the genealogical-guidance system 100 can leverage user-account data to generate and/or dynamically augment a genealogical-research journey for a user account. In some embodiments, a genealogical-research journey includes a set of discrete functions that, when executed, achieve a genealogical-research target. In additional or alternative embodiments, a genealogical-research journey can also include a journey-progress indicator and/or one or more advancing functions.

Relatedly, the term “genealogical-research target” refers to a research target (or goal) associated with a genealogical-research journey for a user account within a genealogical-research (or genealogical-content) system. In particular, a genealogical-research target may include or refer to a target (or goal) related to genealogical research for a user account to achieve upon executing a set of discrete functions. As an example, a genealogical-research target may be a goal (or target) associated with a genealogical or genealogy tree (e.g., a family tree or pedigree), such as to build a genealogy tree, to modify a genealogy tree by adding or removing nodes, and/or to add information to a genealogy tree. As another example, a genealogical-research target may be a goal (or target) associated with genomic data (e.g., DNA), such as utilizing DNA to explore ancestral origins, ethnicity, migration patterns, and/or genomic connections. Other examples of genealogical-research targets include searching for or discovering genealogical content items or records or determining information about a specific individual. In some cases, the genealogical-guidance system 100 can determine a genealogical-research target in response to user input defining the genealogical-research target via a client device. For example, the user may input their genealogical-research target in a pertinent field of a graphical user interface displayed on a client device as free-form text. In other embodiments, the user may be prompted, via the graphical user interface, to select one or more of a predetermined slate of genealogical-research targets, such as “Learn more about how my ancestors migrated,” “Learn more about what my ancestors looked like,” “Find stories about my ancestors’ participation in historical events,” “Learn about milestones in my ancestors’ lives,” “Trace my roots as far back as possible,” and the like. In the same or other embodiments, the genealogical-guidance system 100 can determine (or predict) a genealogical-research target, such as by generating a prediction as to a likelihood that a user account is targeting a particular goal or task through its usage of data and tools within a genealogical-data system (e.g., based on user-account data and user interactions). Such predictions may be performed by any suitable modality, including machine-learned classifiers, heuristics, or otherwise.

As also used herein, the term “function” (e.g., in a set of discrete functions) refers to segment of computer code or a subroutine that is executable by a processor to generate, from input data, an output as part of a genealogical-research journey. In particular, a function can be executed by a genealogical-data system to progress towards (and/or to achieve) a genealogical-research target. For example, a function may be guidance that corresponds to or includes one or more hints, assistance, suggestions, recommendations, directions, and/or an automated execution for a user account to, for example, consider, accept (or reject), address, follow, interact with, and/or execute. Further, functions can be included within the guidance scaffolding. In some embodiments, a function applies (or calls) an orchestrator (e.g., a journey orchestration model) to agentically access and/or utilize a particular skill set, applies (or calls) an Application Programming Interface (“API”), executes a heuristic code segment, generates a notification for display on a client device, extracts specific data from one or more databases (e.g., database 106), and/or the like.

In at least one embodiment, a function can be at least one advancing function. As used herein, the term “advancing function” refers to a discrete function among a set of discrete functions that advances or progresses a genealogical-research journey. Specifically, an advancing function is a discrete function that can, upon execution, advance (or progress) the genealogical-research journey towards achieving the genealogical-research target. In some embodiments, the genealogical-guidance system 100 determines an advancing function in response to detecting a journey metric. Further, in the same or other embodiments, the genealogical-guidance system 100 provides a notification and/or visualization (e.g., a hint, suggestion, or recommendation, such as a widget) of (or associated with) an advancing function for display on a client device associated with a user account.

Additionally, as used herein, the term “user-account data” refers to data (or information) associated with a user account within a genealogical-data system. Specifically, user-account data can be data, associated with a user account, which the genealogical-guidance system 100 can analyze to personalize a genealogical-research journey for the user account. For instance, the genealogical-guidance system 100 can extract (or receive) user-account data associated from a user account and can analyze (or process) the user-account data. Based on this analysis, the genealogical-guidance system 100 may utilize the user-account data in various aspects and/or processes associated with a genealogical-research journey. In some cases, the user-account data can include user experiences, user interactions, user skill sets, user-account settings, user-account content, and/or levels of engagement associated with the user account in relation to various genealogical databases, tools, and interfaces of a genealogical-data system.

Further, as used herein, the term “journey-progress indicator” refers to an indicator and/or memory of progress made in and/or context of one or more genealogical-research journeys. Specifically, the journey-progress indicator can include or refer to a computer code segment, digital marker, and/or memory storage that designates and/or stores one or more histories of actions taken and/or a subset of discrete functions from among a set of discrete functions that are (or have been) executed along or during one or more genealogical-research journeys, including a most recently executed function and/or a next-to-be-executed discrete function among a set of discrete functions that make up a genealogical-research journey. In some embodiments, the genealogical-guidance system 100 can utilize one or more journey-progress indicators to concurrently generate, track, categorize, store, and/or restore (or resume) data associated with one or more genealogical-research journeys associated with a single user account.

As also used herein, the term “journey metric” can refer to a metric indicating when a step has been taken (or has not been taken) to start, progress in, and/or to seek guidance (or assistance) in a genealogical-research journey. For instance, a journey metric can include an action or an inaction (e.g., non-progression in a genealogical-research journey), a determined genealogical-research target, an interaction with a journey orchestration model, and/or an execution of a discrete function (e.g., an advancing function) in the set of discrete functions. In some embodiments, the genealogical-guidance system 100 determines an advancing function in response to detecting a journey metric.

Additionally, as used herein, the term “guidance scaffolding” refers to an underlying framework utilized by (and/or incorporated within) the genealogical-guidance system 100. In particular, the guidance scaffolding can be the underlying framework defining a structure of databases and models that supports and/or orchestrates a genealogical-research journey. For instance, the guidance scaffolding may drive the progression of genealogical-research tasks (e.g., to determine an advancing function) and/or may provide structured assistance, decision-making processes, and/or automation capabilities. In one or more embodiments, the guidance scaffolding may include product integrations, where the guidance scaffolding integrates one or more products of a genealogical-data system. In at least one embodiment, the guidance scaffolding includes one or more of the following: a personalization engine, a journey-progress indicator, an orchestrator (e.g., a journey orchestration model), a set of discrete functions, and/or mechanisms for surfacing a research-target element.

Moreover, as used herein, the term “orchestrator” or “journey orchestration model” refers to a model (or multiple models), such as a generative model, configured to assist a user account along or during a genealogical research journey. In one or more embodiments, an orchestrator includes or corresponds to one or more AI models such as one or more machine learning models (e.g., large language models) and/or agentic models, application programming interfaces (APIs), data models, and/or heuristics models configured to agentically access, determine, select, and/or utilize one or more skill sets for determining and/or executing functions (e.g., advancing functions) specific to a skill or data type. Specifically, based on user-account data, one or more journey-progress indicators, and/or one or more sets of discrete functions, an orchestrator can agentically access, determine, select, and/or utilize skill sets (or models, such as large language models) that can include a help skill set, a discovery skill set, a personalized-guidance skill set, and/or a tree-assistant skill set. In the same or other embodiments, an orchestrator can agentically access, determine, select, and/or utilize additional or alternative skill sets or models. Further, in at least one embodiment, an orchestrator includes, corresponds to, or utilizes an AI assistant provided for display via a GUI of a client device, where one or more user accounts may interact with the AI assistant (e.g., via a user query such as a text input) and receive responses.

As used herein, the term “machine learning model” refers to a computer algorithm or a collection of computer algorithms that automatically improve for a particular task through iterative outputs or predictions based on use of data. For example, machine learning model can utilize one or more learning techniques to improve in accuracy and/or effectiveness. Example machine learning models include various types of neural networks, decision trees, support vector machines, and Bayesian networks. In some embodiments, the model modification system utilizes a machine learning model in the form of a neural network and/or a large language model.

Relatedly, as used herein, the term “neural network” refers to a machine learning model that can be trained and/or tuned based on inputs to determine classifications, scores, or approximate unknown functions. For example, a neural network includes a model of interconnected artificial neurons (e.g., organized in layers) that communicate and learn to approximate complex functions and generate outputs based on a plurality of inputs provided to the neural network. In some cases, a neural network refers to an algorithm (or set of algorithms) that implements deep learning techniques to model high-level abstractions in data. A neural network can include various layers such as an input layer, one or more hidden layers, and an output layer that each perform tasks for processing data. For example, a neural network can include a deep neural network, a convolutional neural network, a recurrent neural network (e.g., an LSTM), a graph neural network, a transformer neural network, or a generative adversarial neural network. Upon training as described below, such a neural network may become a large language model that generates responses to prompts by interpreting prompt language, accessing additional data from content items, and executing functions indicated by prompts and/or content items.

As used herein, the term “generative model” refers to a machine learning model that is configured to generate new data samples that are statistically consistent with the data on which the model was trained. Generative models may include, but are not limited to, large language models, diffusion models, variational autoencoders, generative adversarial networks, and multimodal transformer models. Generative models may be configured to generate outputs across various modalities, including text, images, audio, and video, either independently or conditionally based on input data from the same or a different modality. For example, a generative model may generate an image based on a textual prompt, generate speech based on textual input, or generate text based on prior conversational context, and may operate using probabilistic or iterative denoising methods to produce high-quality, contextually appropriate outputs.

Along these lines, as used herein, the term “large language model” (or “LLM”) refers to a set of one or more machine learning models trained to perform computer tasks to generate or identify computing code and/or data in response to trigger events (e.g., user interactions, such as text queries and button selections). In particular, a large language model can be a neural network (e.g., a deep neural network) with many parameters trained on large quantities of data (e.g., unlabeled text) using a particular learning technique (e.g., self-supervised learning). For example, a large language model can include parameters trained to generate or identify computing code and/or data based on various contextual data, including information from historical user account behavior.

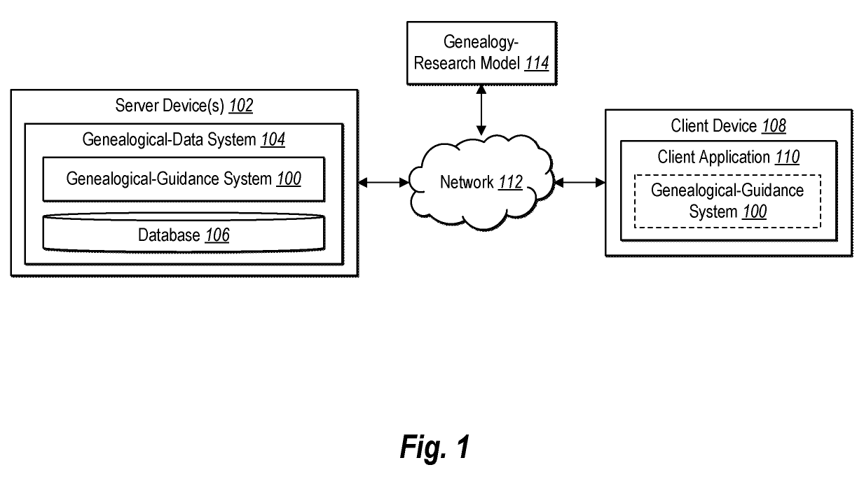

Additional detail regarding the genealogical-guidance system 100 will now be provided with reference to the figures. For example, FIG. 1 illustrates a schematic diagram of an example system environment for implementing the genealogical-guidance system 100 in accordance with one or more embodiments. An overview of the genealogical-guidance system 100 is described in relation to FIG. 1. Thereafter, a more detailed description of the components and processes of the genealogical-guidance system 100 is provided in relation to the subsequent figures.

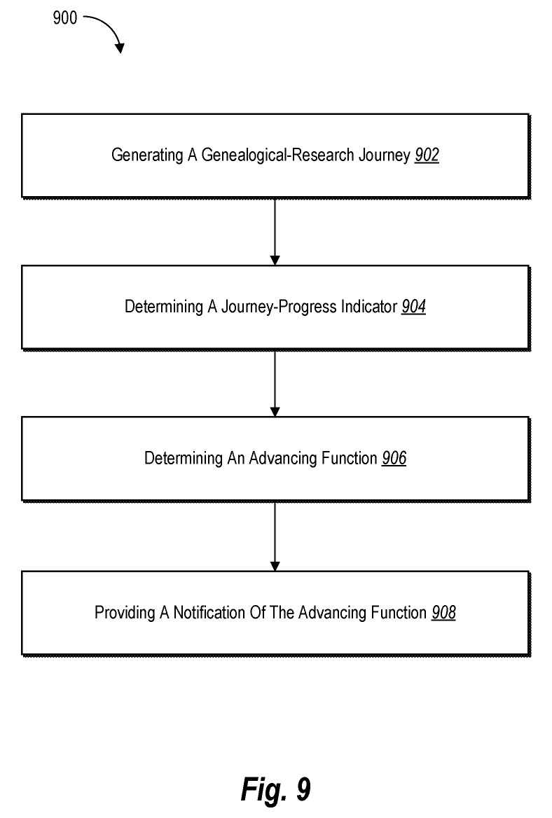

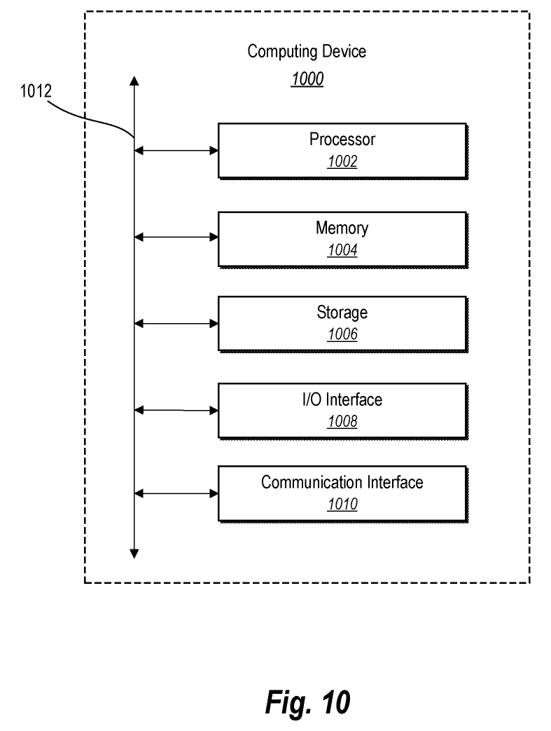

As shown, the environment includes server device(s) 102, a client device 108, a database 106, a network 112, and a genealogy-research model 114. Each of the components of the environment can communicate via the network 112, and the network 112 may be any suitable network over which computing devices can communicate. Example networks are discussed in more detail below in relation to FIGS. 9-10.

As mentioned above, the example environment includes a client device 108. The client device 108 can be one of a variety of computing devices, including a smartphone, a tablet, a smart television, a desktop computer, a laptop computer, a virtual reality device, an augmented reality device, or another computing device as described in relation to FIGS. 9-10. The client device 108 can communicate with the server device(s) 102 and/or the database 106 via the network 112. For example, the client device 108 can receive user input from respective users interacting with the client device 108 (e.g., via the client application 110) to, for instance, search for, identify, access, generate, modify, or share genealogical-research journey data, user-account data, DNA data, and/or genealogical data (e.g., genealogy tree data and/or genealogical record data) via a graphical user interface of the genealogical-data system 104. In addition, the genealogical-guidance system 100 on the server device(s) 102 can receive information relating to various searches for, or interactions with, genealogical-research journey data, user-account data, DNA data, genealogical data, and/or user interface elements based on the input received by the client device 108.

As shown, the client device 108 can include a client application 110. In particular, the client application 110 may be a web application, a native application installed on the client device 108 (e.g., a mobile application, a desktop application, etc.), or a cloud-based application where all or part of the functionality is performed by the server device(s) 102. Based on instructions from the client application 110, the client device 108 can present or display information, including a user interface such as a genealogical record search interface, a genealogical journey interface, or some other graphical user interface, as described herein.

As illustrated in FIG. 1, the example environment also includes the server device(s) 102. The server device(s) 102 may generate, track, store, process, receive, and transmit electronic data, such as search requests, genealogical records, record queries, query results, genealogical-research journey data, user-account data, DNA data, and/or genealogical data. For example, the server device(s) 102 may receive data from the client device 108 in the form of an indication of a selection to view a particular graphical user interface or to perform a search for a genealogical record. In addition, the server device(s) 102 can transmit data to the client device 108 within a graphical user interface. Indeed, the server device(s) 102 can communicate with the client device 108 to send and/or receive data via the network 112. In some implementations, the server device(s) 102 comprise(s) a distributed server where the server device(s) 102 include(s) a number of server devices distributed across the network 112 and located in different physical locations. The server device(s) 102 can comprise one or more content servers, application servers, communication servers, web-hosting servers, machine learning servers, and other types of servers.

As shown in FIG. 1, the server device(s) 102 can also include the genealogical-guidance system 100 as part of a genealogical-data system 104. The genealogical-data system 104 can communicate with the client device 108 to perform various functions associated with the client application 110 such as determining genealogical-research targets; generating genealogical-research journeys; determining advancing functions; managing genealogical-research journey data, user accounts and user-account data, DNA data, genealogical data, and/or genealogical records; and/or facilitating user interaction with, and sharing of, genealogical records. Indeed, the genealogical-data system 104 can include a network-based cloud storage system to manage, store, and maintain genealogical-research journey data, user-account data, DNA data, genealogical data, and/or genealogical records. For instance, the genealogical-data system 104 can utilize genealogical data across various content items and user accounts to generate and maintain a universal genealogy tree that reflects the relatedness or consanguinity between nodes corresponding to all user accounts and other individuals indicated by stored genealogical records. In some embodiments, the genealogical-guidance system 100 and/or the genealogical-data system 104 utilize the database 106 to store and access information such as genealogical-research journey data, user-account data, DNA data, genealogical data, genealogical records, genealogy trees, and/or other information.

As further illustrated in FIG. 1, a genealogy-research model 114 can interact with the network 112. In particular, the genealogy-research model 114 can leverage the network 112 to transmit and/or receive data to and from the server device(s) 102 and/or the client device 108. In one or more embodiments, the genealogy-research model 114 includes guidance scaffolding utilized by the genealogical-guidance system 100, and can transmit and/or receive data associated with the guidance scaffolding. For instance, the genealogy-research model 114 can transmit and/or receive data that includes genealogical-research journey data, user-account data, DNA data, genealogical data, and/or other personalization data associated with one or more user accounts.

Although FIG. 1 depicts the genealogical-guidance system 100 located on the server device(s) 102, in some implementations, the genealogical-guidance system 100 may be implemented by (e.g., located entirely or in part on) one or more other components of the environment. For example, the genealogical-guidance system 100 may be implemented in whole or in part by the client device 108. For example, the client device 108 and/or a third-party system can download all or part of the genealogical-guidance system 100 for implementation independent of, or together with, the server device(s) 102.

In some implementations, though not illustrated in FIG. 1, the environment may have a different arrangement of components and/or may have a different number or set of components altogether. For example, the client device 108 may communicate directly with the genealogical-guidance system 100, bypassing the network 112. As another example, the environment may include multiple client devices, each associated with a different user account. In addition, the environment can include the database 106 located external to the server device(s) 102 (e.g., in communication via the network 112) or located on the server device(s) 102 and/or on the client device 108.

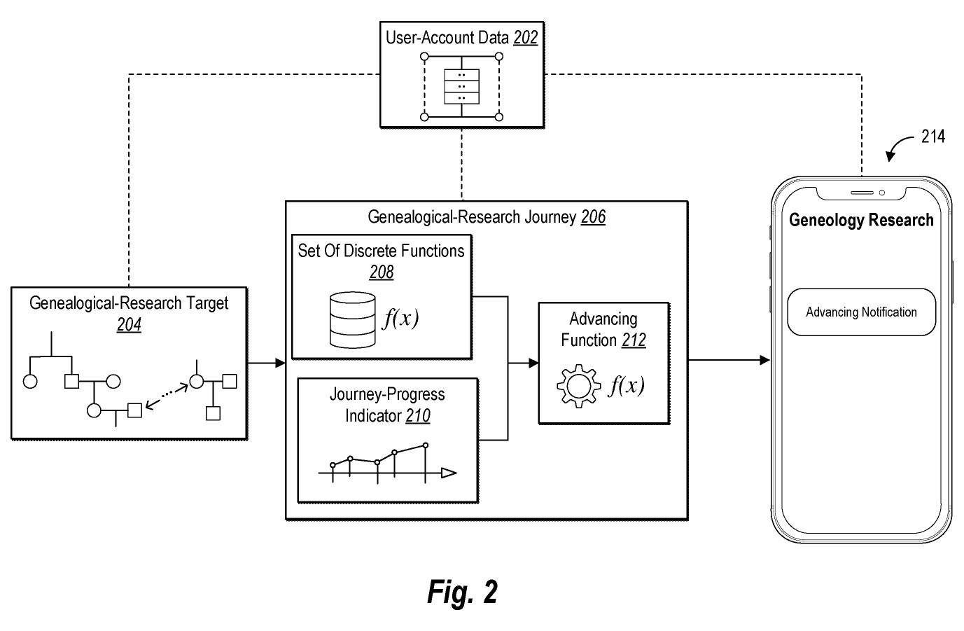

As expressed above, the genealogical-guidance system 100 can determine an advancing function. In particular, the genealogical-guidance system 100 can determine an advancing function for a genealogical-research journey based on user-account data, a set of discrete functions, and/or a journey-progress indicator. FIG. 2 illustrates an example diagram of determining an advancing function for a genealogical-research journey and providing a notification of the advancing function to a client device in accordance with one or more embodiments. Additional detail regarding the acts and processes introduced in relation to FIG. 2 is provided thereafter with reference to subsequent figures. In one or more embodiments, the genealogical-guidance system 100 determines (and/or progresses through) a genealogical-research journey 206 utilizing a particular user interface and/or guidance scaffolding that facilitates integration of one or more guidance models and/or processes.

As illustrated in FIG. 2, the genealogical-guidance system 100 utilizes the user-account data 202 in various aspects and/or processes associated with a genealogical-research journey 206. In particular, the genealogical-guidance system 100 may extract (or receive) user-account data 202 associated with a user account within the genealogical-data system 104. In some instances, the genealogical-guidance system 100 analyzes (or processes) the user-account data 202 (e.g., upon extracting or receiving it).

Based on this analysis, the genealogical-guidance system 100 can utilize the user-account data 202 to personalize one or more aspects and/or processes associated with a genealogical-research journey 206. Indeed, the genealogical-guidance system 100 can utilize a personalization engine to personalize the one or more aspects and/or processes. For example, the genealogical-guidance system 100 utilizes the user-account data 202 (and, in some cases, the personalization engine) to personalize a genealogical-research target 204, personalize a genealogical-research journey 206, and/or provide or receive data (e.g., an advancing notification) to or from a client device 214. In the same or other embodiments, the user-account data 202 is also utilized to personalize additional or alternative aspects and/or processes associated with a genealogical-research journey 206, including journey metrics and guidance scaffolding utilized by (or included within) the genealogical-guidance system 100 (as explained above). In some cases, the user-account data can include user experience, user skill sets, user-account settings, user-account content, and/or levels of engagement associated with the user account.

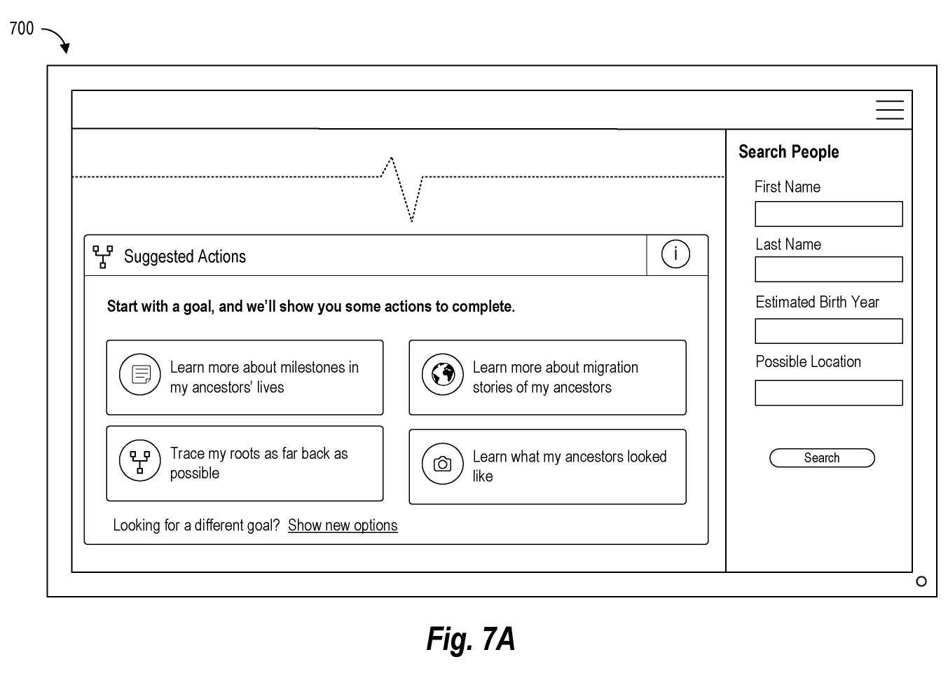

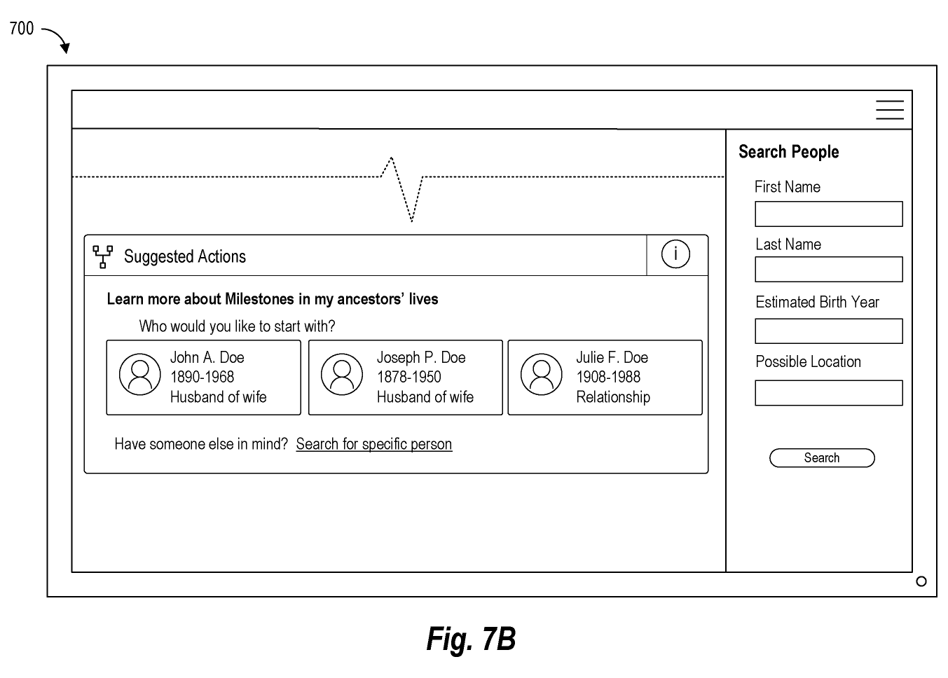

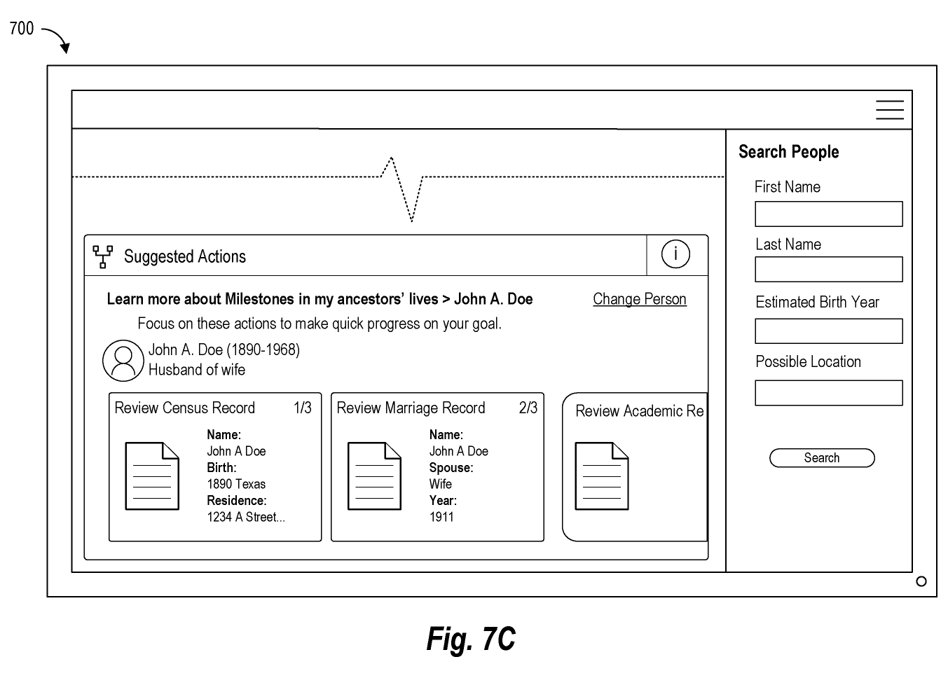

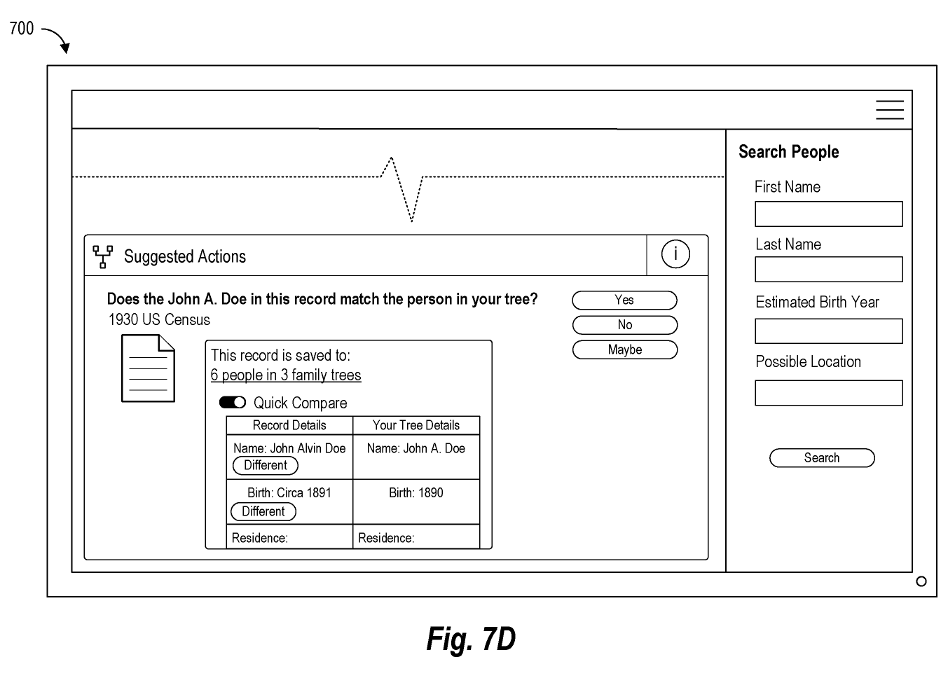

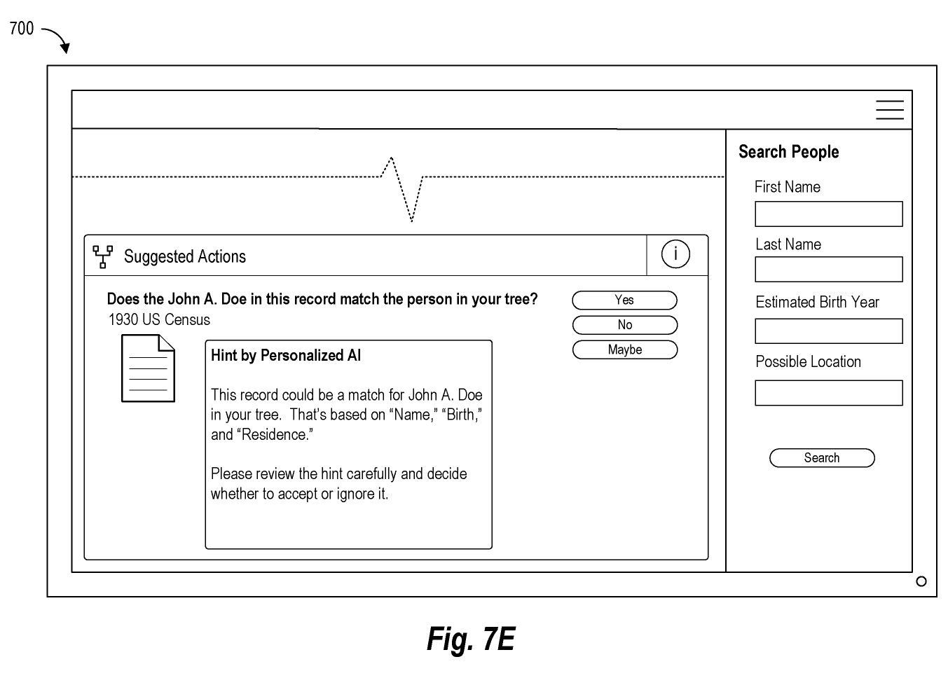

As also shown in FIG. 2, the genealogical-guidance system 100 determines the genealogical-research target 204. For instance, the genealogical-guidance system 100 may determine the genealogical-research target 204 in response to detecting user input or user interaction corresponding to a client device expressly defining the genealogical-research target 204 (e.g., via a user query in an agent interface or via a user selection of one or more predetermined genealogical-research targets, as seen in at least FIG. 7A). In some embodiments, the genealogical-guidance system 100 can determine (or predict) the genealogical-research target 204 in an automated fashion (e.g., without an express user input defining the genealogical-research target 204) based on user-account data 202 and interactions with various data and tools of the genealogical-data system 104. For instance, the genealogical-guidance system 100 may determine the genealogical-research target 204 by generating a prediction of a likelihood or a probability that the user account is performing actions progressing towards achieving a particular genealogical-research target. In one or more embodiments, the genealogical-guidance system 100 determines the genealogical-research target 204 based on the user-account data 202. In additional or alternative embodiments, the genealogical-guidance system 100 can determine a plurality of genealogical-research targets for the user account.

As further illustrated in FIG. 2, the genealogical-guidance system 100 generates the genealogical-research journey 206 for the user account based on the determined genealogical-research target 204. In particular, the genealogical-guidance system 100 can generate the genealogical-research journey 206 to provide structured, personalized guidance aimed at assisting the user account to progress towards (or to achieve) the genealogical-research target 204. For instance, the genealogical-guidance system 100 leverages the user-account data 202 to generate the genealogical-research journey 206. In one or more embodiments, the genealogical-research journey 206 includes (but is not limited to) a set of discrete functions 208, a journey-progress indicator 210, and/or an advancing function 212. In some instances, the genealogical-guidance system 100 can generate a plurality of genealogical-research journeys (e.g., for each respective genealogical-research target of the plurality of genealogical-research targets), each with their own set of discrete functions, journey-progress indicators, and/or advancing functions.

As just mentioned, the genealogical-research journey 206 can include the set of discrete functions 208. In particular, the genealogical-guidance system 100 determines the set of discrete functions 208 for the genealogical-research journey 206 based on the user-account data 202 and/or the genealogical-research target 204. For instance, the genealogical-guidance system 100 leverages the user-account data 202 and the genealogical-research target 204 to determine which discrete functions of, for example, a plurality of preexisting discrete functions available within the genealogical-data system 104, will guide the user account along or dring the genealogical-research journey 206 towards achieving the genealogical-research target 204. The genealogical-guidance system 100 can then aggregate (or combine) these determined discrete functions into the set of discrete functions 208.

As mentioned above, the genealogical-research journey 206 can include the journey-progress indicator 210. Specifically, the genealogical-guidance system 100 can determine the journey-progress indicator 210 for the genealogical-research journey 206 based on the user-account data 202 and/or the genealogical-research target 204. In some cases, the genealogical-guidance system 100 utilizes the journey-progress indicator 210 to generate and store a record of discrete functions in the set of discrete functions 208 that are executed in the genealogical-research journey 206. By utilizing the journey-progress indicator 210, the genealogical-guidance system 100 can restore and continue (or resume) the genealogical-research journey 206 at the point (e.g., at the discrete function in the set of discrete functions 208) in the genealogical-research journey 206 where the journey was paused and/or otherwise discontinued (e.g., when another genealogical-research journey is initiated or continued).

As mentioned above, the genealogical-research journey 206 can include the advancing function 212. In particular, the genealogical-guidance system 100 determine the advancing function 212 based on the journey-progress indicator 210. For instance, based on the journey-progress indicator 210, the genealogical-guidance system 100 can determine a discrete function from among the set of discrete functions 208 which may, upon execution, progress the genealogical-research journey 206 towards achieving the genealogical-research target 204. Based on this determination, the genealogical-guidance system 100 may select that discrete function to be the advancing function 212. In some instances, the genealogical-guidance system 100 determines a plurality of advancing functions.

Additionally, as shown in FIG. 2, the genealogical-guidance system 100 provides a notification (e.g., an advancing notification) of the determined advancing function 212 to a client device 214 associated with the user account. In particular, upon determining the advancing function 212, the genealogical-guidance system 100 can provide the notification of the advancing function 212 for display in a graphical user interface (GUI) of the client device 214. In the same or other embodiments, the genealogical-guidance system 100 may provide one or more elements with the advancing notification. In some cases, the one or more elements can be selectable to accept, decline, and/or otherwise interact with the advancing notification (e.g., to access genealogical content, crawl a genealogy tree, confirm that an individual in a first genealogical record corresponds to a second individual in a genealogy tree, etc.).

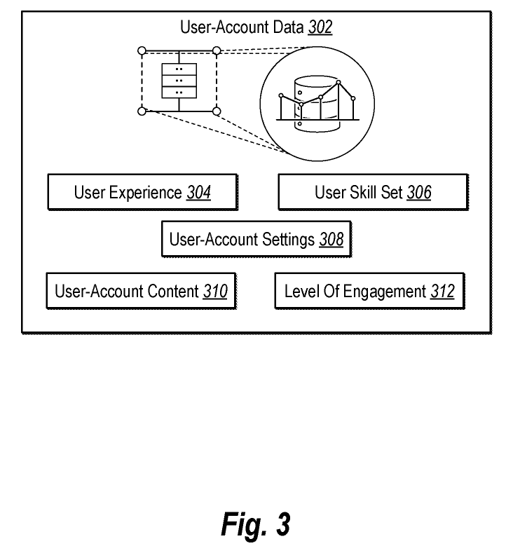

As expressed above, the genealogical-guidance system 100 utilizes user-account data to personalize a genealogical-research journey for a particular user account. For instance, the genealogical-guidance system 100 can leverage the user-account data to personalize various aspects of the genealogical-research journey, including determining a genealogical-research target for the genealogical-research journey, determining a set of discrete functions for the genealogical-research journey, and/or determining an advancing function from among the set of discrete functions. FIG. 3 illustrates an example diagram of analyzing user-account data to personalize a genealogical-research journey for a user account in accordance with one or more embodiments.

As illustrated in FIG. 3, the genealogical-guidance system 100 accesses, extracts, receives, or otherwise obtains a user-account data 302 associated with a user account of the genealogical-data system 104. For instance, the genealogical-guidance system 100 accesses, extracts, receives, or otherwise obtains the user-account data 302 from one or more servers of the genealogical-data system 104 and/or from one or more servers located external to the genealogical-data system 104. In at least one embodiment, the user-account data 302 includes one or more of user experience 304 (or user experience data), a user skill set 306 (or user skill set data), user-account settings 308, user-account content 310, and/or a level of engagement 312. In at least one embodiment, the genealogical-guidance system 100 utilizes the user-account data 302 in various aspects and/or processes associated with a genealogical-research journey to personalize that journey, as described in greater detail above and below.

As just mentioned, the user-account data 302 includes the user experience 304 of the user account. To elaborate, in at least some instances, the user experience 304 includes information describing the user’s familiarity with genealogical research tools, genealogical concepts, and/or the genealogical-data system 104. For example, the user experience 304 can include data indicating whether a user is a novice, intermediate, or advanced genealogical researcher, a duration of time the user has interacted with the genealogical-data system, a quantity of genealogical research journeys initiated or completed by the user account, and/or a number of advancing functions executed by the user.

As also mentioned above, the user-account data 302 can include the user skill set 306 of the user account. To illustrate, the user skill set 306 can include information describing particular genealogical skills, research capabilities, proficiencies, and/or competencies associated with the user account. For example, the user skill set 306 can include data indicating whether the user is proficient in genealogical skills such as interpreting genealogical records, analyzing DNA matches, constructing or editing family trees, identifying historical records, evaluating potential ancestor connections, and/or resolving conflicting genealogical information. In at least one embodiment, the genealogical-guidance system 100 determines, for the user account, one or more skill scores for one or more genealogical skills and/or for the user account as a whole. For example, the genealogical-guidance system 100 can determine the one or more skill scores and/or the user skill set 306 based on explicit inputs provided by the user (e.g., the user account indicating one or more user skill sets) and/or inferred behaviors associated with the user account, such as previously executed advancing functions, completed genealogical research journeys, or interactions with particular genealogical records.

Additionally, as mentioned above, the user-account data 302 can include the user-account settings 308 associated with the user account. For example, the user-account settings 308 include configurable preferences and parameters associated with the user account, such as preference or parameters that influence how the genealogical-data system 104 operates for the user. In particular, the user-account settings 308 can include preferences relating to notification settings, privacy settings, language preferences, display preferences, and/or preferences relating to genealogical research guidance and/or genealogical research journeys. In some embodiments, the user-account settings 308 can also include settings specifying how genealogical research journeys are presented or executed, which types of genealogical records are analyzed or prioritized, and/or how recommendations or advancing functions are delivered to the user account.

Further, as mentioned above, the user-account data 302 can include user-account content 310 associated with the user account. Specifically, the user-account content 310 can include a quantity and/or coverage of genealogical content associated with the user account included within a user-account database (e.g., of the genealogical-data system 104 and/or located externally to the genealogical-data system 104). For example, the user-account content 310 can include information relating to a structure of a genealogical or genealogy tree (e.g., a family tree or pedigree) associated with the user account, a quantity of nodes (e.g., entities or individuals) within the genealgocial tree, a number of edges or relationships defined between nodes, and/or an extent to which genealogical records or metadata have been attached to the nodes within the genealogy tree. In some embodiments, the user-account content 310 can additionally or alternatively include information describing growth of the genealogy tree over time, completeness of genealogical profiles, genomic data (e.g., DNA) associated one or more individuals, and/or a volume of genealogical records, documents, or sources associated with the user account.

Moreover, as mentioned above, the user-account data 302 can include the level of engagement 312 of the user account. To elaborate, the level of engagement 312 can include a measure or indication of engagement with which the user account has had with the genealogical-data system 104. For example, the level of engagement 312 can include information relating to a frequency with which the user account accesses the genealogical-data system 104, a duration of user sessions, a quantity of interactions performed within the genealogical-data system 104, and/or a number of genealogical research journeys initiated, resumed, and/or completed by the user account. In some embodiments, the level of engagement 312 additionally or alternatively includes information describing user account interactions with one or more particular features, tools, and/or products of the genealogical-data system 104, such as a personalized guidance feature and/or other genealogical research feature or tool.

In at least one embodiment, the user-account data 302 includes one or more additional or alternative forms or types of user-account data associated with the user account, such as a history of behavior (or behavioral data). For example, history of behavior can include information describing prior interactions of the user account with the genealogical-data system 104, including previously executed advancing functions, navigation patterns within genealogical research journeys, selections of suggested records or hints, and/or modifications made to genealogical content and/or genealogy trees. In some embodiments, the history of behavior can further include sequences of actions performed by the user account over time, abandoned or incomplete genealogical research journeys, and/or patterns indicating preferences or tendencies of the user account.

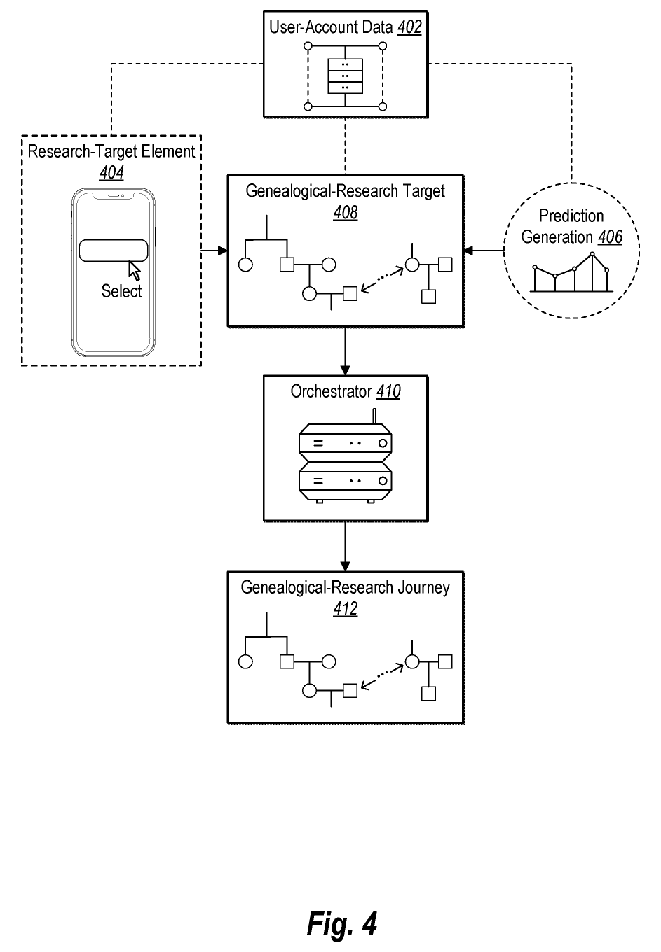

As expressed above, the genealogical-guidance system 100 can generate a genealogical research journey. For instance, the genealogical-guidance system 100 determines a genealogical-research target and generates a genealogical-research journey for progressing towards or achieving the genealogical-research target. FIG. 4 illustrates an example diagram of generating a genealogical-research journey to achieve a genealogical-research target in accordance with one or more embodiments. Further, FIG. 4 illustrates user-account data 402, a predefined research-target element 404, an act 406 of prediction generation, a genealogical-research target 408, an orchestrator 410 (e.g., a journey orchestration model), and the genealogical-research journey 412.

As illustrated in FIG. 4, the genealogical-guidance system 100 utilizes the user-account data 402 associated with a user account for one or more aspects and/or processes associated with the genealogical-research journey 412 to personalize the journey. Indeed, the genealogical-guidance system 100 can utilize a personalization engine to process the user-account data 402 and to personalize the one or more aspects and/or processes. For instance, the genealogical-guidance system 100 utilizes the user-account data 402 (and, in some cases, the personalization engine) in generating and providing, for display in a client device, one or more predetermined research-target elements (e.g., the predefined research-target element 404), performing the act 406 of prediction generation, determining the genealogical-research target 408, and/or in progressing through the genealogical-research journey 412 toward the genealogical-research target 408. In at least one embodiment, the genealogical-guidance system 100 accesses, extracts, receives, or otherwise obtains the user-account data 402 as described above (e.g., in relation to FIG. 3).

As also illustrated in FIG. 4, the genealogical-guidance system 100 determines the genealogical-research target 408. To elaborate, as shown, the genealogical-guidance system 100 can determine the genealogical-research target 408 in response to a user interaction or selection of the predefined research-target element 404. In the same or other embodiments, the genealogical-guidance system 100 determines the genealogical-research target 408 based on performing the act 406 of prediction generation.

In one or more embodiments, the genealogical-guidance system 100 determines whether to provide one or more predetermined (or standard) research-target elements and/or whether to perform the act 406 based on the user-account data 402 and one or more thresholds (e.g., data thresholds). For instance, to make this determination, the genealogical-guidance system 100 compares the user-account data 402 to the one or more thresholds. As an example, the one or more thresholds may correspond to an amount of data included in the user-account data 302, such as an amount of user experience, the presence or absence of a particular user skill, particular user-account settings, particular types or amounts of user-account content, and/or an amount of the level of user engagement. In at least one embodiment, if the user-account data 402 satisfies the one or more thresholds, the genealogical-guidance system 100 performs the act 406. In the same or other embodiments, if the user-account data 402 fails to satisfy the one or more thresholds, the genealogical-guidance system 100 generates and provides one or more predetermined research-target elements.

As just mentioned, if the user-account data 402 fails to satisfy the one or more thresholds, the genealogical-guidance system 100 can generate and provide the one or more predetermined research-target elements. In one or more embodiments, the genealogical-guidance system 100 generates and provides the one or more predetermined research-target elements as graphical elements for display via a GUI of the client device. In at least one embodiment, the one or more predetermined research-target elements correspond to or include standard-path or predetermined sets of discrete functions for achieving particular genealogical-research targets.

As also mentioned, if the user-account data 402 satisfies the one or more thresholds, the genealogical-guidance system 100 can perform the act 406 of prediction generation, which can enable the genealogical-guidance system 100 to dynamically generate personalized genealogical-research journeys. In particular, the genealogical-guidance system 100 can perform the act 406 by generating one or more predictions of one or more genealogical-research targets that the user account is likely to pursue or progress towards (or may wish to pursue or achieve). For example, the genealogical-guidance system 100 can utilize the user-account data 402 (e.g., a history of behavior of the user account) to generate the one or more predictions.

Based on the one or more predictions, the genealogical-guidance system 100 can automatically select the genealogical-research target 408 (and/or a plurality of genealogical-research targets) from the predicted one or more genealogical-research targets. For example, the genealogical-guidance system 100 may determine one or more likelihoods that the user account will elect to progress towards the one or more genealogical-research targets. As an example, the genealogical-guidance system 100 can generate prediction or propensity scores for the one or more genealogical-research targets and automatically select the genealogical-research target 408 from the predicted one or more genealogical-research targets based on the prediction or propensity scores.

In the same or other embodiments, the genealogical-guidance system 100 generates and provides, to a GUI of the client device, one or more prediction-based research-target elements (or widgets) that indicate or initiate, progress towards, and/or achieve the predicted one or more genealogical-research targets. For example, upon predicting that the user account may wish to pursue or achieve a genealogical-research target of modifying a genealogy tree, the genealogical-guidance system 100 generates a prediction-based research-target element for initiating a genealogical-research journey for modifying the genealogy tree. In one or more embodiments, the genealogical-guidance system 100 determines or selects the genealogical-research target 408 in response to receiving, via a GUI of the client device, a user interaction with a prediction-based research-target element of the one or more prediction-based research-target elements. In at least one embodiment, the genealogical-guidance system 100 generates and provides, for display via the GUI, one or more visual indications or notifications (e.g., widgets) recommending, to the user account, one or more of the predicted genealogical-research targets and/or prediction-based research-target elements (e.g., based on the one or more likelihoods—such as the prediction or propensity scores for the one or more genealogical-research targets—described above).

In the same or other embodiments, the genealogical-guidance system 100 determines the genealogical-research target 408 utilizing an AI model (e.g., an agentic model and/or an LLM), such as the orchestrator 410. For instance, the genealogical-guidance system 100 can determine and/or select the genealogical-research target 408 by performing the act 406 utilizing the AI model. In at least one embodiment, in response to receiving a text query or text input provided via an agent interface, the genealogical-guidance system 100 utilizes the AI model to automatically select the genealogical-research target 408 and/or to generate and/or provide (e.g., in a conversation with the AI model via the GUI) the one or more predefined research-target elements and/or the one or more prediction-based research-target elements based on the text query or text input.

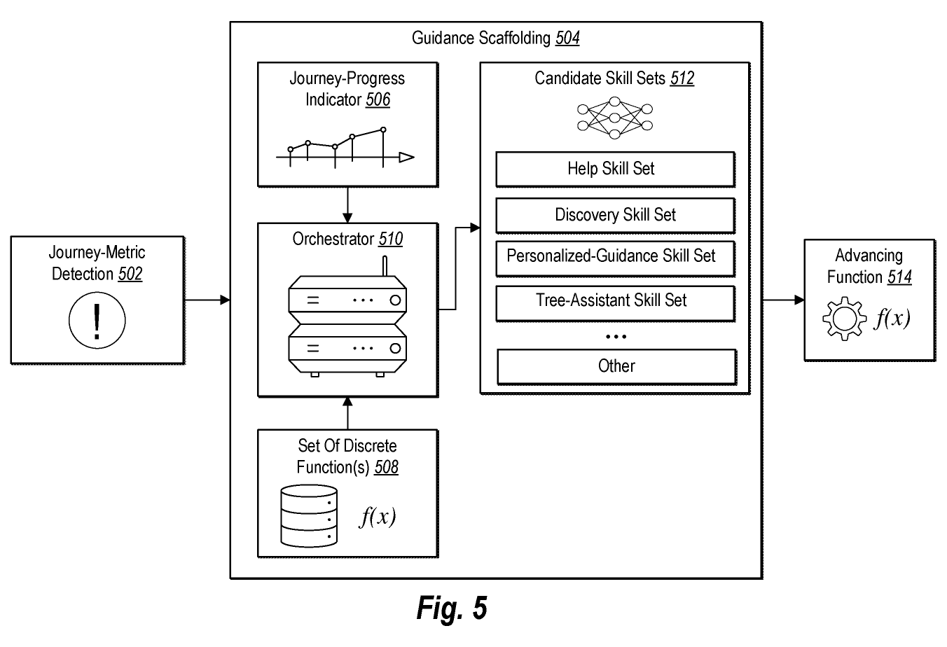

As further illustrated in FIG. 4, in response to selection or determination of the genealogical-research target 408, the genealogical-guidance system 100 utilizes the orchestrator 410 (or journey orchestration model) to guide the user account through the genealogical-research journey 412. For example, the orchestrator 410 can correspond to or include an LLM configured to orchestrate functions (e.g., a set of discrete functions and/or one or more advancing functions) as part of the genealogical-research journey 412. In at least one embodiment, the genealogical-guidance system 100 can utilize the orchestrator 410 as described above and below in greater detail (e.g., in relation to FIG. 5).

As expressed above, the genealogical-guidance system 100 can determine and execute an advancing function to progress a genealogical-research journey toward a genealogical research target. In particular, the genealogical-guidance system 100 can utilize guidance scaffolding that includes a journey-progress indicator, set of discrete functions, an orchestrator, and/or a plurality of candidate skill sets to determine and execute the advancing function. FIG. 5 illustrates an example diagram of utilizing guidance scaffolding to determine an advancing function in accordance with one or more embodiments.

As illustrated, FIG. 5 includes an act 502 of journey-metric detection. As also illustrated, FIG. 5 includes guidance scaffolding 504, which includes a journey-progress indicator 506, a set of discrete functions 508, an orchestrator 510 (e.g., the orchestrator 410), and/or a plurality of candidate skill sets 512. Additionally, as illustrated, FIG. 5 includes an advancing function 514. Further, as shown, the orchestrator 510 can be configured to, based on a journey-progress indicator 506, the act 502, and/or a set of discrete functions 508 (e.g. a set of functionalities of a genealogical research service), determine one or more skill sets from the plurality of candidate skill sets 512 for determining and executing one or more advancing functions along or during a genealogical-research journey (e.g., the genealogical-research journey 412).

To elaborate, as illustrated in FIG. 5, the genealogical-guidance system 100 performs the act 502 of journey-metric detection. For instance, the genealogical-guidance system 100 detects a journey metric indicating an inaction, a determined or selected genealogical-research target (e.g., the genealogical-research target 408), an interaction with the orchestrator 510, and/or an execution of an advancing function. For example, the genealogical-guidance system 100 detects a journey metric indicating an inaction by detecting non-progression (e.g., that the user account is stuck) in the genealogical-research journey, such as for a threshold amount of time and/or user interactions with a client device. Additionally or alternatively, the genealogical-guidance system 100 can detect a journey metric indicating an inaction by predicting, through user-account data analysis, one or more areas or times that one or more user accounts of the genealogical-data system 104 fail to progress (or get stuck). As another example, the genealogical-guidance system 100 detects a journey metric indicating an interaction with the orchestrator 510 in response to detecting a user interaction (e.g., a user query such as a text input) with an AI assistant (e.g., corresponding to or associated with the orchestrator 510) via a GUI of a client device.

As also illustrated in FIG. 5, the genealogical-guidance system 100 utilizes the guidance scaffolding 504 to adapt the genealogical-research journey to the research needs and/or research targets of a user account. For example, the genealogical-guidance system 100 utilizes the guidance scaffolding 504 to support and/or orchestrate the genealogical-research journey by providing structured assistance, decision-making processes, and/or automation capabilities. Additionally or alternatively, the guidance scaffolding 504 may include product integrations, where the guidance scaffolding 504 integrates one or more products of the genealogical-data system 104. For example, the genealogical-guidance system 100 can cause the guidance scaffolding 504 to include a hints product for providing basic recommendations, a genomic (e.g., DNA) journey product that provides genomic information, such as information about ethnicities, to the user account along or during the genealogical-research journey, and/or one or more additional or alternative products of the genealogical-data system 104. In at least one embodiment, the genealogical-guidance system 100 utilizes user-account data (e.g., the user-account data 302) to help the user account to identify one or more ways to interact with the one or more products (e.g., to improve their interactions). As an example, the genealogical-guidance system 100 can help the user account by offering guidance on how to review a hint or other suggestion or recommendation properly, how to improve research based on the hint, etc.

As illustrated in FIG. 5, the guidance scaffolding 504 includes the journey-progress indicator 506. In particular, the genealogical-guidance system 100 can generate, manage, and/or utilize the journey-progress indicator 506 to indicate and/or record progress made in one or more genealogical-research journeys. For example, the genealogical-guidance system 100 utilizes the journey-progress indicator 506 to record a history of a subset of discrete functions (e.g., advancing functions) from one or more sets of discrete functions that are, or have been, executed along or during one or more genealogical-research journeys, including a most recently executed function.

In at least one embodiment, the genealogical-guidance system 100 records the history of performed acts and/or past-executed discrete (or advancing) functions according to one or more thresholds. For example, the one or more thresholds can include a threshold number of past-executed discrete functions (e.g., the 25 most recently performed acts or past-executed discrete functions) and/or a threshold amount of time (e.g., within the past 72 days). Additionally or alternatively, the genealogical-guidance system 100 can utilize the journey-progress indicator 506 to record one or more next-to-be-executed discrete functions from one or more sets of discrete functions (e.g., from one or more genealogical-research journeys). Further, in one or more embodiments, the journey-progress indicator 506 stores, or causes to be stored, actions taken and/or past-executed discrete (or advancing) functions in a linear or sequential manner, or in a non-linear manner (e.g., in a branching or graph-based structure).

Further, as illustrated in FIG. 5, the guidance scaffolding 504 includes the set of discrete functions 508. In particular, the genealogical-guidance system 100 can generate or determine the set of discrete functions 508 that includes one or more discrete functions determined or predicted to assist the user account in progressing towards or achieving the genealogical-research target. In one or more embodiments, the genealogical-guidance system 100 generates or determines the set of discrete functions 508 similar to how the genealogical-guidance system 100 determines or selects the genealogical-research target (e.g., concurrently or sequentially with the genealogical-research target). For example, the genealogical-guidance system 100 generates and/or determines the set of discrete functions 508 in response to a user interaction or selection of one or more predefined research-target elements (e.g., the predefined research-target element 404), as described in greater detail above (e.g., in relation to FIG. 4). In the same or other embodiment, the genealogical-guidance system 100 generates and/or determines the set of discrete functions 508 in response to generating one or more predictions of one or more genealogical-research targets that the user account is likely to pursue or progress towards (e.g., based on performing the act 406), as also described in greater detail above (e.g., in relation to FIG. 4).

Moreover, as illustrated in FIG. 5, the guidance scaffolding 504 includes the orchestrator 510 or journey orchestration model which can correspond to or include an LLM. In at least one embodiment, the genealogical-guidance system 100 enables or causes the orchestrator 510 to receive, access, and/or utilize user-account data, the journey-progress indicator 506, and/or the set of discrete functions 508 and to determine and/or execute the advancing function 514. Indeed, based on the user-account data, the journey-progress indicator 506, and/or the set of discrete functions 508, the genealogical-guidance system 100 can cause the orchestrator 510 to agentically determine and/or select one or more skill sets from the plurality of candidate skill sets 512 that may be (e.g., that is predicted to be) well-suited for determining and/or executing the advancing function 514. For example, based on performing the act 502 which may take into account user-account data (e.g., user activity, interests, goals, genealogy trees, media, DNA results, or otherwise), the genealogical-guidance system 100 can utilize the orchestrator 510 to determine that the personalized-guidance skill set may be well (or most) suited to generating the advancing function 514.

In at least some instances, the one or more skill sets can focus on one or more specific tasks, objectives, contexts, and/or problem domains when determining (or predicting) and/or executing the advancing function 514. For instance, the genealogical-guidance system 100 can cause the orchestrator 510 to receive data from the journey-progress indicator 506 and/or the set of discrete functions 508. Upon receiving this data, the genealogical-guidance system 100 can cause the orchestrator 510 to process the data and determine which skill set(s) of the plurality of candidate skill sets 512 could be used to determine (or would be effective in determining) the advancing function 514.

As illustrated in FIG. 5, the plurality of candidate skill sets 512 include one or more of the help skill set or universal genealogical-help skill set, the discover skill, the personalized-guidance skill set, the tree-assistant skill set, and/or one or more additional or alternative skill sets. For instance, the genealogical-guidance system 100 causes the orchestrator 510 to utilize the help skill set to answer user queries generally related or broadly applicable to genealogical information, genealogical research, and/or the genealogical-data system 104. For example, the genealogical-guidance system 100 causes the orchestrator 510 to utilize the help skill set to generate and provide, to the client device, one or more response to one or more user queries received from the user account. In one or more embodiments, the genealogical-guidance system 100 causes the orchestrator 510 to utilize a retrieval-augmented generation (RAG) approach to receive or extract context and/or content items from a vector database for generating a broadly applicable genealogy-research response. In some instances, the RAG approach includes utilizing an embeddings model, a vector database, and/or a large language model (“LLM”) to receive or extract the context and/or content items.

In the same or other embodiments, the genealogical-guidance system 100 causes the orchestrator 510 to utilize the discovery skill set to discover additional or alternative genealogical content or information. For instance, the genealogical-guidance system 100 causes the orchestrator 510 to utilize the discovery skill set to discover genealogical content or information that can be incorporated or included into storage associated with the user account. As an example, the genealogical-guidance system 100 causes the orchestrator 510 to provide one or more hints that guide the user account to genealogical content or information and/or provide genealogical information to the user account. In at least one embodiment, the additional or alternative genealogical content or information can be information to include into a genealogy tree (e.g., family tree) or other genealogical data structure or content item. Further, in some embodiments, the genealogical-guidance system 100 causes the orchestrator 510 to utilize a RAG and/or retrieval-augmented thought approach to generate user-specific responses to user queries or hints. In some instances, the RAG and/or retrieval-augmented thought approach include the use of an embeddings model, one or more vector databases, and/or other modalities as suitable for generating chain-of-thought prompting for an LLM configured to generate user-specific responses.

In the same or other embodiments, the genealogical-guidance system 100 causes the orchestrator 510 to utilize the personalized-guidance skill set to provide personalized guidance for the user account to progress along one or more genealogical-research journeys. To illustrate, the genealogical-guidance system 100 causes the orchestrator 510 to receive inputs of user account data, including such things as a user’s profile, activity on the genealogical research service including the user’s engagement with various functionalities thereof, records, genealogy trees, or DNA results associated with the user, and/or any other suitable input of user-account data. Further, the genealogical-guidance system 100 can cause the orchestrator 510 to receive the one or more genealogical-research targets pursued by the user account (whether predicted on behalf of the user or expressed by the user). In response to receiving these inputs, the genealogical-guidance system 100 causes the orchestrator 510 to generate, using an LLM or any other suitable modality, a suggested next-best action or advancing function for progressing in a genealogical-research journey and/or achieving one or more genealogical-research targets.

Moreover, the genealogical-guidance system 100 causes the orchestrator 510 to utilize the personalized-guidance skill set to response to personalized user queries associated with one or more user accounts, such as a request for guidance for the genealogical-research journey. For example, the genealogical-guidance system 100 can detect and/or receive a user interaction (e.g., an input of a user query or prompt) with an AI assistant (e.g., corresponding to or associated with the orchestrator 510) included in a GUI of the client device, where the user interaction is relevant to (or associated with) the user account. In response, the genealogical-guidance system 100 can cause the orchestrator 510 to utilize the personalized-guidance skill set to generate a personalized response to the user interaction that can guide the user account to a next action or recommend a next action to the user account, generate and/or provide one or more content items or answers, and/or automatically determine and/or execute one or more advancing functions.

For example, the genealogical-guidance system 100 can receive one or more user queries or prompts such as, “How do I get more hints for how to proceed?”, “Tell me how to trace my roots (or family tree) as far back as possible,” and/or “How can I expand my family tree?” In response, the genealogical-guidance system 100 causes the orchestrator 510 to utilize the personalized-guidance skill set to generate and provide a personalized response, which can include one or more selectable elements for obtaining additional information and/or executing one or more advancing functions. As an example, in response to a user query, the genealogical-guidance system 100 can utilize the orchestrator 510 to generate a response providing one or more hints, providing one or more pathways for exploring genomic data (e.g., DNA), and/or indicating or recommending one or more nodes (e.g., entities) that the user account could look into including in a genealogy tree.

In at least one embodiment, the personalized-guidance skill set is additionally or alternatively configured to cooperate with a machine-learned model or modality configured to generate a next-best node for a user to explore using a neural network and based on input features such as content-level genealogical metrics, tree-level genealogical metrics, and account-level genealogical metrics as well as historical records, tree persons determined to be potentially related using the cluster database, or other hintable content. While a neural network has been described, it will be appreciated that heuristic approaches may additionally or alternatively be relied upon. Such a neural network may be the neural network described in U.S. Patent Application Publication No. 2024/0378252, published Nov. 14, 2024, which is hereby incorporated by reference in its entirety.

In the same or other embodiments, the genealogical-guidance system 100 causes the orchestrator 510 to utilize the tree-assistant skill set to crawl, expand, and/or generate one or more genealogy trees. In particular, the genealogical-guidance system 100 can cause the orchestrator 510 to utilize the tree-assistant skill set to crawl a genealogy tree, map the genealogy tree, identify existing relationships (e.g., via edges and/or metadata) based on the genealogy tree, predict new relationships based on the genealogy tree, and/or respond to queries associated with the genealogy tree. In at least one embodiment, the tree-assistant skill set is configured to receive or access, as one or more inputs, a genealogy tree (e.g., a family tree or extant pedigree) of a user account and and/or additional or alternative user-account data.

Further, the tree-assistant skill set can configured to determine, using an LLM or other suitable modality, a map of the genealogy tree, one or more relationships in (or missing from) the genealogy tree, and/or an identification of mismatches between existing nodes and/or media or data attached thereto (e.g., on the basis of historical-record data from historical records and/or based on the aforementioned cluster database). Additionally or alternatively, the tree-assistant skill set can be configured to generate biographical details about a genealogy tree and/or ancestors. Further, in the same or other embodiments, the tree-assistant skill set can be configured to utilize one or more APIs to determine one or more candidate nodes corresponding to one or more entities to include, or recommend including (e.g., in a hint), into a genealogy tree. As an example, the genealogical-guidance system 100 can cause the orchestrator 510 to, via the tree-assistant skill set and the one or more APIs, generate recommendation scores (or hint scores) for the one or more entities and to include, or recommend including, at least one of the one or more entities into the genealogy tree based on the recommendation scores.

In at least one embodiment, the genealogical-guidance system 100 utilizes the orchestrator and/or one or more of the plurality of candidate skill sets 512 to determine and execute the advancing function 514 (by, e.g., expanding a genealogy tree of the user account, adding pertinent or high-quality historical records to nodes of the genealogy tree, or otherwise). In one or more instances, the genealogical-guidance system 100 provides a notification and/or a visualization (e.g., a widget) of, or associated with, the advancing function 514 for display via the GUI of the client device. Further, the notification and/or visualization of the advancing function can include or correspond to a hint, suggestion, and/or recommendation for the user account to follow and/or to initiate execution of the advancing function 514 or an additional or alternative advancing function.

In one or more embodiments, in cases where the genealogical-guidance system 100 causes the orchestrator 510 to determine and/or execute the advancing function 514, the genealogical-guidance system 100 updates, modifies, and/or improves the journey-progress indicator 506. For example, in response to detecting that the advancing function 514 has been executed, the genealogical-guidance system 100 can modify or update the journey-progress indicator 506 to record the execution of that advancing function. Additionally, in some instances, the genealogical-guidance system 100 utilizes the orchestrator 510 (and/or one or more of the plurality of candidate skill sets 512) to determine and/or execute one or more additional advancing functions for the genealogical-research journey. In the same or other embodiments, upon detecting the execution of the advancing function 514, the genealogical-guidance system 100 determines that the genealogical-research target has been met and concludes the corresponding genealogical-research journey. Moreover, in some instances, the genealogical-guidance system 100 can generate and provide, to the client device, a notification to the user account indicating that the genealogical-research target has been met and/or that the corresponding genealogical-research journey has concluded.

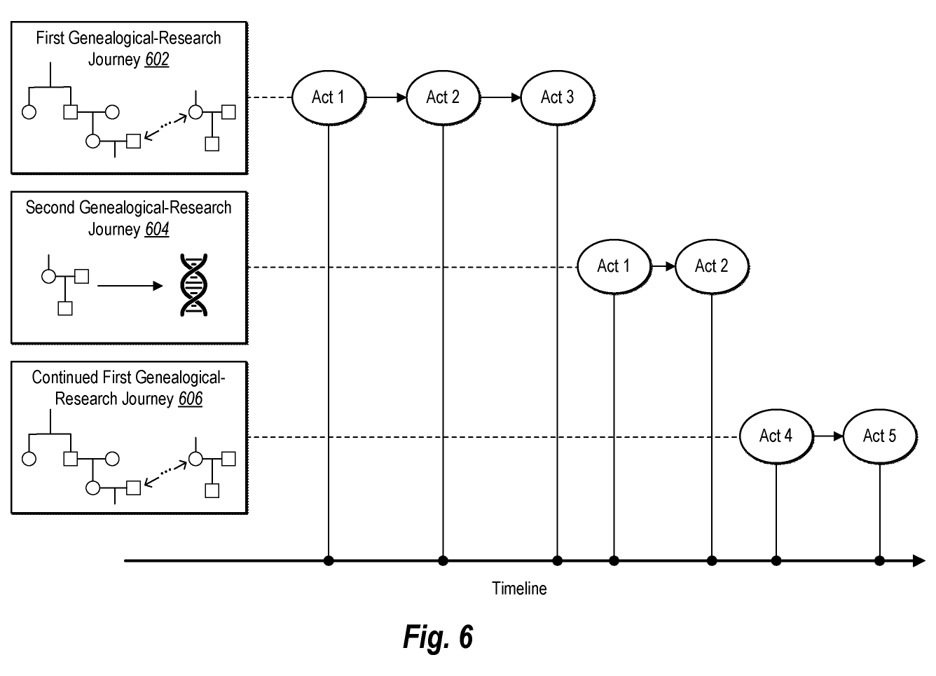

As expressed above, the genealogical-guidance system 100 can generate a plurality of genealogical-research journeys for achieving one or more genealogical-research targets. In at least one embodiment, the genealogical-guidance system 100 utilizes one or more journey-progress indicators to pause or discontinue and resume or continue one or more of the plurality of genealogical-research journeys. FIG. 6 illustrates an example timeline of various acts performed in a first genealogical-research journey and in a second genealogical-research journey in accordance with one or more embodiments. As illustrated, FIG. 6 includes a first genealogical-research journey 602, a second genealogical-research journey 604, and a continued first genealogical-research journey 606.

As illustrated in FIG. 6, the genealogical-guidance system 100 can pause or discontinue a genealogical-research journey and resume or continue that journey at a later point in time. For example, the genealogical-guidance system 100 can enable pausing of the first genealogical-research journey 602, followed by initiating or continuing the second genealogical-research journey 604, followed by reverting from the second genealogical-research journey 604 back to the first genealogical-research journey 602 (e.g., the continued first genealogical-research journey 606). In one or more instances, the genealogical-guidance system 100 generates the second genealogical-research journey 604 that diverges from the genealogical research journey and that includes a second set of discrete functions executable by the genealogical-data system 104. In at least one embodiment, the genealogical-guidance system 100 can monitor, record, and/or manage a plurality of genealogical-research journeys (e.g., the first genealogical-research journey 602 and the second genealogical-research journey 604) utilizing a journey-progress indicator, as described above in greater detail (e.g., in relation to FIG. 5).

Indeed, as illustrated in FIG. 6, the genealogical-guidance system 100 detects interruptions of the first genealogical-research journey 602 and/or a return to, or resuming of, the first genealogical-research journey 602. As an example, upon performing or executing one or more acts or advancing functions (e.g., acts 1, 2, and 3) of the first genealogical-research journey 602, the genealogical-guidance system 100 detects an interruption of the first genealogical-research journey 602 by detecting a pause or discontinuation (e.g., in response to a user interaction with a client device) of the first genealogical-research journey 602 and/or by detecting a continuation or initiation (or beginning) of the second genealogical-research journey 604. Additionally or alternatively, upon performing or executing one or more acts or advancing functions (e.g., acts 1 and 2) and/or upon completing the second set of discrete functions of the second genealogical-research journey 604, the genealogical-guidance system 100 reverts back to the first genealogical-research journey 602 (e.g., the continued first genealogical-research journey 606).

For instance, the genealogical-guidance system 100 reverts back to the first genealogical-research journey 602 upon detecting completion of the second genealogical-research journey 604 and/or upon detecting an interruption of the second genealogical-research journey 604. As an example, the genealogical-guidance system 100 detects the interruption of the second genealogical-research research journey by detecting a pause or discontinuation (e.g., in response to a user interaction with a client device) of the second genealogical-research journey 604 and/or by detecting a continuation of the first genealogical-research journey 602. Upon reverting back to or resuming the first genealogical-research journey 602 (e.g., the continued first genealogical-research journey 606), the genealogical-guidance system 100 can perform or execute one or more additional acts or advancing functions (e.g., Acts 4 and 5).

In one or more embodiments, the genealogical-guidance system 100 stores details from one or more of the genealogical-research journeys 602, 604, 606 in one or more databases of the genealogical-data system 104 and/or utilize the details as context for generating and/or recommending one or more additional or alternative advancing functions. For example, the genealogical-guidance system 100 can store details from the genealogical-research journeys 602, 604, 606 utilizing an API or by other suitable means such as by accessing user-activity parameters from the genealogical-data system 104. Further, the genealogical-guidance system 100 can store the details to preserve context between and among the genealogical-research journeys 602, 604, 606. By so doing, the genealogical-guidance system 100 advantageously determines improved advancing functions for a user in view of past and present activity and interests while capitalizing on resource economies by leveraging previously generated and/or executed advancing functions, user responses thereto, and/or retrieved historical records, entities associated with a genealogy tree, etc.

In at least one embodiment, the genealogical-guidance system 100 is configured to store a predetermined number of user interactions (e.g. a past 25 acts or executed advancing functions of one or a plurality of genealogical-research journeys) in memory for a predetermined length of time (e.g., 72 days) to preserve context for subsequent genealogical-research acts or advancing functions. In some instances, the genealogical-guidance system 100 maintains such memory as a rolling or constantly updating cache of previous acts and/or executed advancing functions.

As expressed above, the genealogical-guidance system 100 determines one or more genealogical-research targets and/or generates one or more genealogical-research journeys for progressing towards or achieving the one or more genealogical-research targets. In some instances, the genealogical-guidance system 100 determines the one or more genealogical-research targets in response to a user interaction via a GUI of a client device. FIGS. 7A-7E illustrate example user interfaces of a client device for determining a genealogical-research target and executing various advancing functions in accordance with one or more embodiments.

As illustrated, FIGS. 7A-7E include a graphical user interface (GUI) 700 (or a plurality of GUIs)—such as genealogical-research journey interface—and progression along a genealogical-research journey. For instance, FIGS. 7A-7E illustrated a user account selecting one or more genealogical-research targets and determining and/or executing one or more advancing functions and/or visualizations (e.g., hints, suggestions, recommendations, etc., such as one or more widgets) associated with the one or more advancing functions.

As illustrated in FIG. 7A, a user account accesses, via a client device, a genealogical research service associated with the genealogical-data system 104, which may be displayed to the user on the GUI 700. The GUI 700 may include a section or a modal that includes one or more avenues or methods for activating or initiating one or more genealogical-research journeys. For instance, the genealogical-guidance system 100 can enable activation or initiation of a genealogical-research journey via a specific (e.g., a main or home) page of the genealogical research service and/or via interaction with an AI assistant (e.g., corresponding to or associated with an orchestrator).

To elaborate, as illustrated in FIG. 7A, the GUI 700 may include a plurality of suggested actions (e.g., via one or more selectable elements) corresponding to one or more genealogical research targets, which can activate or initiate one or more genealogical-research journeys towards achieving the one or more genealogical research targets. For example, the plurality of suggested actions may include learning more about milestones in the life of one or more ancestors, learning more about migration stories of one or more ancestors, tracing one’s roots back as far as possible, learning what one or more ancestors looked like, etc. Where none of the displayed suggested actions are appealing to a user, the user may select an option for showing one or more additional or alternative (e.g., new) options corresponding to one or more additional or alternative genealogical research targets. The plurality of suggested actions may be provided in a predetermined number (e.g., four), but the disclosure is not limited thereto. Further, the genealogical-guidance system 100 can modify or change the plurality of suggested actions displayed via the GUI 700.

In one or more embodiments, the genealogical-guidance system 100 can generate (e.g., utilizing the genealogical research service) the plurality of suggested actions described above. For example, the genealogical-guidance system 100 can utilize certain biographical details about a user, such as a new user of the genealogical research service, to determine the plurality of suggested actions. For existing users, the plurality of suggested actions for one or more genealogical-research targets may be based on user-account data as described above (e.g., in relation to FIG. 3), including, e.g., history of behavior, user experience data, user skill set(s) data, user-account settings, user-account content, level of engagement, etc. For instance, the genealogical-guidance system 100 can cause an orchestrator to utilize one or more skill sets to retrieve or extract a constructed genealogy tree(s) of a user account—along with associated tree persons or entities and attached media, search histories and selections, DNA results such as detected DNA communities, ethnicities, traits, matches, or otherwise, or other details. As an example, the genealogical-guidance system 100 can cause the orchestrator to utilize the a help skill set, a discovery skill set, a personalized-guidance skill set, a tree-assistant skill set, or any other suitable skill set or modal, to generate a journey-progress indicator and/or to determine and execute one or more advancing functions, as described in greater detail above (e.g., in relation to FIG. 5).

To illustrate, as shown in FIGS. 7A-7E, the genealogical-guidance system 100 may provide, to a user account, one or more widgets or graphical elements shown by means of the GUI 700. The one or more widgets may utilize or be configured to cooperate with a backend scaffolding, as discussed herein, of integrations with existing services and functions of the genealogical research service, such as DNA-results engines (e.g. functionalities for generating an estimate, based on a genotyping dataset received for a user, of phased haplotypes, associated ethnicities or communities, phenotypes, matches, etc.), a cluster database with clusters of tree persons appearing in different trees, historical-records databases and associated search modalities therefor, and/or any other suitable modality. As such, the personalized-guidance embodiments may be configured to send and receive data, including user-interaction data, with a plurality of APIs or tools associated with a plurality of existing functionalities.

In one or more embodiments, while the GUI 700 is shown in a logged-in home page of the genealogical research service, it will be appreciated that one or more embodiments may be applied within a page or tool specific to any of the above-mentioned functionalities, such as within a DNA-results functionality of the genealogical research service. Thus, the personalized-guidance widget may be integrated with existing functionalities of the genealogical research service in a variety of manners, such as by a dedicated widget in a home page of the genealogical research service or as part of an existing, specific functionality thereof. Further, the backend scaffolding may be configured to utilize or cooperate with a journey orchestration model that can utilize one or more skill sets, as described above (e.g., in relation to FIG. 5).

In at least one embodiment, the genealogical-guidance system 100 generates and provides a hint-assistant model configured to generate a summary of a generated hint (e.g., generated utilizing the journey orchestration model) that a user has not taken action on. For example, the genealogical-guidance system 100 can utilize the hint-assistant model to generate a summary of a generated hint by, for example, attaching a historical record to a tree node, and/or updating data associated with a tree node based on a hint comparison, etc.) to encourage the user based on the likelihood of accuracy or pertinence of the hint and the associated historical record or data to take action thereon.

As illustrated in FIG. 7B, in response to a user interaction with one or more of the plurality of suggested actions and/or one or more associated selectable elements such as, “Learn more about milestones in my ancestors’ lives,” the genealogical-guidance system 100 generates and provides a subsequent section, page, GUI, or modal of the genealogical research service. For example, the genealogical-guidance system 100 may determine which ancestors of the user are most likely to yield interesting milestone-related information or stories and populate a predetermined number of said ancestors. The genealogical-guidance system 100 can provide or present such ancestors in the GUI 700 along with one or more selectable elements, along with, in some cases, identifying information such as name, birth and death year, and relationship information. Should a user not be interested in searching any of the presented ancestors, the genealogical-guidance system 100 enables a user account to select one or more additional or alternative selectable element for showing one or more additional or alternative ancestors or for searching for a specific person. Additionally, or alternatively, a search modality may be presented to the user in a sidebar (as illustrated in FIGS. 7A–7E) or in any other suitable manner.

As illustrated in FIG. 7C, upon a user account selecting a particular ancestor (e.g., John A. Doe), the genealogical-guidance system 100, via the GUI 700, provides or presents a plurality of specific actions or advancing functions specific to learning about milestones in the life of the particular ancestor. The genealogical-guidance system 100 can provide the plurality of specific actions or advancing functions in a predetermined number of cards that a user may swipe, scroll, or otherwise peruse. As an example, the plurality of specific actions or advancing functions may include reviewing historical records (e.g., census records, marriage records, academic records, etc.), publicly available genealogy tree nodes or clusters, or other actions or functions available via connected tools of the genealogical research service. For instance, the genealogical-guidance system 100 may prompt a user account to review a census record, a marriage record, and/or an academic record in which the genealogical-guidance system 100 has determined or predicted that the particular ancestor appears in or is associated with. In at least one embodiment, the genealogical-guidance system 100 ranks the presented specific actions and/or advancing functions based on a determined likelihood of progressing towards one or more genealogical-research targets or in any other suitable manner, such as based on a heuristic determined from most-selected or most-engaged-with types of media or other resources across the genealogical research service.

As illustrated in FIG. 7D, upon a user selecting one of the presented specific actions or advancing functions such as reviewing the census record, the genealogical-guidance system 100 generates and provides (e.g., populates) details from the census record may be populated in the GUI 700 (e.g., for expeditious review by the user). In at least one embodiment, upon selection by a user (or based on default section) of a “quick compare” toggle or option, the GUI 700 populates a comparison of pertinent details extracted by the genealogical research service from the census record with details from a node corresponding to the selected ancestor in a genealogy tree, allowing for the user to quickly and intuitively determine whether there are any errors in a genealogy tree based on the census record and/or whether the census record does, in fact, pertain to the user’s selected ancestor.

In at least one embodiment, the genealogical-guidance system 100 can determine, or can enable the user to indicate via one or more selectable elements, whether the census record does, in fact, pertain to a person in the genealogy tree. In response to determining that the census record does pertain to a person in the genealogy tree such as in response to a user interaction with a selectable element (e.g., “Yes”), the genealogical-guidance system 100 can facilitate convenient attachment of the census record as a content item to the corresponding node of the genealogy tree. Additionally, the genealogical-guidance system 100 can access and utilize one or more cluster databases of the genealogical-data system 104 to provide one or more indicia of the reliability of the record, such as that the census record is saved or attached to six people in three family trees across the genealogical-data system 104.

As an addition or an alternative to the “quick compare” toggle or option shown in FIG. 7D, a user may select (or the genealogical research service may select or default to) a full comparison view, where the user may select the record to enter a view mode of the record. The genealogical-guidance system 100 can guide the user account in the GUI 700 to a pertinent section of the census record, for example, with, in embodiments, fields corresponding to the user’s ancestor automatedly highlighted or otherwise modified (e.g., by the genealogical-guidance system 100) to facilitate the user’s perusal of the historical record for pertinence. In one or more embodiments where the selected record is, for example, a historical newspaper image, the genealogical-guidance system 100 can highlight or segment the image to highlight the sections of the newspaper image determined to be pertinent to the selected ancestor for the user’s review.

Additionally or alternatively, as illustrated in FIG. 7E, the genealogical-guidance system 100 generates and provides using one or more of the models or modalities described herein, a summary regarding the selected record. For example, the genealogical-guidance system 100 can provide the record in near-real time or in advance (such as when ranking the records for presentation in the graphical user interface) to a journey orchestration model for assessing the record against user-account data (e.g., the genealogy tree, including tree nodes, tree edges defining relationships between nodes, media such as historical records attached to the tree nodes, and/or clusters of a cluster database to which one or more of the tree nodes have been resolved). The genealogical-guidance system 100 can generate and provide, for display via the GUI 700, the user a generated summary indicating a hint or assessment of whether the presented and selected record indeed pertains to the presented and selected ancestor.

By providing the options, flow, and features shown and described regarding FIGS. 7A–7E, the genealogical-guidance system 100 can guide a user—particularly a new user who has not yet acquired deep genealogical-research skills and expertise, and about whom large volumes of behavioral data and with whom usable data (such as large, established genealogy trees with associated media) have not yet been established for use by predictive modalities—toward specific genealogical-research targets via specific actions or advancing functions that will further their genealogical-research goals (e.g., via a genealogical research service). The genealogical-guidance system 100 advantageously provides personalized guidance to the user on the basis of user-account data and/or on the basis of expressed user preferences (e.g., when faced with a paucity of user-account data)—such as a selection by the user from among a plurality of possible genealogical-research journeys. Accordingly, the genealogical-guidance system 100 enables users to intuitively and expeditiously advance along a genealogical-research journey, benefitting both the user—who receives the benefit of improved emotional engagement with their personal discoveries—and the genealogical-guidance system 100. Indeed, the genealogical-guidance system 100 can receive the benefit of lower load and demand on services, such as record-search services, as the user more-quickly identifies pertinent records to add to pertinent tree nodes.

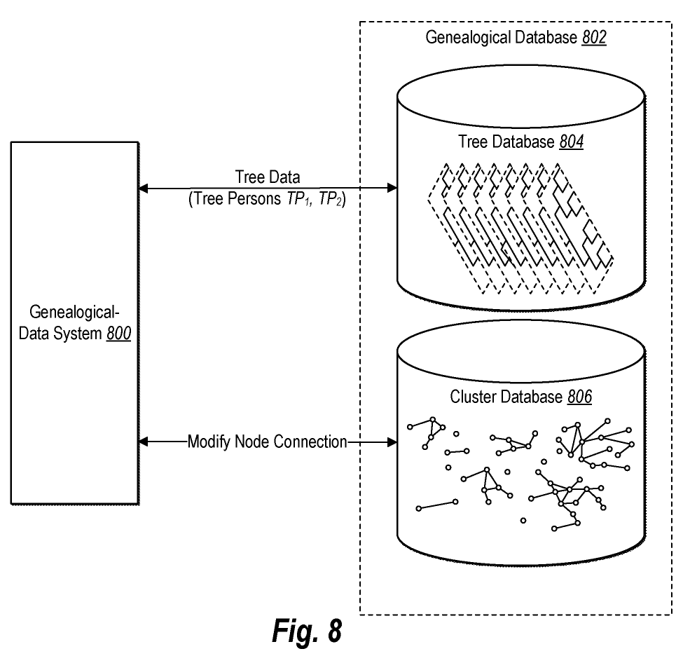

FIG. 8 illustrates a genealogical-data system 800 (e.g., the genealogical-data system 104) interfacing with a genealogical database 802 in accordance with one or more embodiments. For certain genealogical databases, the genealogical-data system 800 identifies groups of user nodes or records in the format of a genealogical tree or records connected by biological and other family relationships as “tree data.” The genealogical-data system 800 can thus search and process tree data stored in a genealogical database 802 (which includes a tree database 804 and a cluster database 806) to execute tasks and perform functions as described herein.

In one or more embodiments, the genealogical-data system 800 can resolve duplicate entities corresponding to respective genealogical records. Indeed, the genealogical-data system 800 can determine that two entities in a cluster database are the same individual, despite differences in various data, such as name spelling, discrepancies in certain dates, and/or other variances in data. The genealogical-data system 800 can analyze clusters of genealogical records stored for each individual within the cluster database 806 to determine that the clusters are within a threshold similarity of one another and that, therefore, the clusters should be combined or otherwise resolved to represent a single individual. In some cases, the genealogical-data system 800 can compare clusters by extracting vectors from the genealogical records in the clusters, averaging the vectors to generate cluster vectors (or otherwise determining weighted or unweighted cluster centers) representative of respective clusters, and determining distances (e.g., Euclidean or cosine distances) between the cluster vectors. The genealogical-data system 800 can further propagate such entity resolution to the tree database 804 to update nodes and edges within a universal genealogical tree, resolving two previously disparate nodes into a single node as indicated by the newly resolved (or combined clusters in the cluster database 806).

For the genealogical database 802, the genealogical-data system 800 may receive genealogical data (e.g., data records and/or genealogical data objects) for building tree data from a source selected from a ground-truth genealogical tree generated from genealogical records and trees of user accounts within the genealogical-data system 800, from the Ancestry World Tree system, a Social Security Death Index database, the World Family Tree system, a birth certificate database, a death certificate database, a marriage certificate database, an adoption database, a draft registration database, a veterans database, a military database, a property records database, a census database, a voter registration database, a phone database, an address database, a newspaper database, an immigration database, a family history records database, a local history records database, a business registration database, and/or a motor vehicle database. Additionally, genealogical data can be user-generated. Genealogical data may also include data from a cluster database 806 derived from records and user data.

Some embodiments of the genealogical-guidance system 100 relate to modifying a cluster database 806 based on a user query and/or other interaction with the genealogical-guidance system 100. In some instances, the genealogical-data system 800 (or the genealogical-guidance system 100) determines and/or modifies a node connection for an individual represented by or resolved to a cluster within the cluster database 806. Indeed, the genealogical-guidance system 100 can analyze, add, remove, and/or modify genealogical content items organized into clusters within the cluster database 806 based on relatedness corresponding to a common individual. The genealogical-guidance system 100 can also access, modify, and analyze genealogical trees within the tree database 804 by, for example, adding nodes, removing nodes, and/or modifying nodes based genealogical content items (and their relationships to individuals) stored within the cluster database 806.

As seen in FIG. 8, the genealogical-data system 800 includes a genealogical database 802, which may include a tree database 804 and a cluster database 806. The tree database 804 may be configured to facilitate the generation, storage, and collation of family trees for a plurality of users, with trees comprising nodes and edges therebetween. Data and records, such as images, may be associated with individual nodes of the trees in the tree database 804. Tree person data, including data such as names, relationships, dates, events, and other metadata may be provided by the tree database 804 to the genealogical-data system 800. The cluster database 806 may include one or more clusters comprising resolved entities, where tree persons (nodes) in different trees in the tree database 804 are associated together in a cluster after determination that the tree persons correspond to a same person.

As a user expands their family tree, e.g. by tagging a previously unknown person in an image using the suggestions provided by the ancestor-identification system and adding the now-identified person to their tree as a new node, the tree database 804 may be modified as the user’s family tree is expanded, and the cluster database 806 may be modified to include the new node in the pertinent cluster. Further, the genealogical-guidance system 100 can attach a conversation with a user account (e.g., a query received from the user account and a response the genealogical-guidance system 100 generates, and/or a series of queries and corresponding responses) to a cluster within the cluster database 806 and/or a node within the tree database 804 to utilize as a ground-truth genealogical tree for future operations within the genealogical-data system 800. For example, the genealogical-guidance system 100 can extract or otherwise pull contextual data from the user account or conversations with the user account and attach the context to a node or a cluster of the cluster database 806 to utilize as a ground-truth genealogical tree for future operations within the genealogical-data system 800 and/or the genealogical-guidance system 100.

FIGS. 1-8, the corresponding text, and the examples provide a number of different systems and methods for generating a genealogical-research journey, determining an advancing function which progresses the genealogical-research journey toward a genealogical-research target upon execution, and providing a notification of the advancing function. In addition to the foregoing, implementations can also be described in terms of flowcharts comprising acts steps in a method for accomplishing a particular result. For example, FIG. 9 illustrates an example series of acts for providing a notification of an advancing function.

While FIG. 9 illustrates acts according to certain implementations, alternative implementations may omit, add to, reorder, and/or modify any of the acts shown in FIG. 9. The acts of FIG. 9 can be performed as part of a method (e.g., a computer-implemented method). Alternatively, a non-transitory computer-readable medium can comprise instructions, that when executed by one or more processors, cause a computing device to perform the acts of FIG. 9. In still further implementations, a system can perform the acts of FIG. 9.

As illustrated in FIG. 9, the series of acts 900 includes an act 902 of generating a genealogical-research journey. In particular, the act 902 involves generating, for a user account within a genealogical-data system, a genealogical-research journey comprising a set of discrete functions executable by the genealogical-data system, the genealogical-research journey achieving a genealogical-research target upon execution of the set of discrete functions. In addition, the series of acts 900 includes an act 904 of determining a journey-progress indicator. In particular, the act 904 involves determining, based on user-account data, a journey-progress indicator defining a subset of discrete functions from the set of discrete functions executable by the genealogical-data system during the genealogical-research journey. As shown, the series of acts 900 includes an act 906 of determining an advancing function. For example, the act 906 involves determining, based on the journey-progress indicator, an advancing function which progresses the genealogical-research journey toward the genealogical-research target upon execution. Further, the series of acts 900 includes an act 908 of providing a notification of the advancing function. For instance, the act 908 involves providing a notification of the advancing function for display on a client device associated with the user account.

For example, the series of acts 900 includes acts to perform any of the operations described in the following clauses: CLAUSE 1. A computer-implemented method comprising: generating, for a user account within a genealogical-data system, a genealogical-research journey comprising a set of discrete functions executable by the genealogical-data system, the genealogical-research journey achieving a genealogical-research target upon execution of the set of discrete functions; determining, based on user-account data, a journey-progress indicator defining a subset of discrete functions from the set of discrete functions executable by the genealogical-data system during the genealogical-research journey; determining, based on the journey-progress indicator, an advancing function which progresses the genealogical-research journey toward the genealogical-research target upon execution; and providing a notification of the advancing function for display on a client device associated with the user account.

CLAUSE 2: The computer-implemented method of clause 1, further comprising determining the user-account data based on at least one of the following: a level of engagement of the user account of the genealogical-data system with the genealogical-data system; user-account content associated with the user account comprised within a user-account database; and a user skill set associated with the user account.

CLAUSE 3: The computer-implemented method of any of clauses 1-2, further comprising: generating a plurality of candidate genealogical-research targets based on the user-account data; and determining the genealogical-research target in response to receiving, from the client device, a user interaction corresponding to a particular genealogical-research target of the plurality of candidate genealogical-research targets.

CLAUSE 4: The computer-implemented method of any of clauses 1-3, wherein generating the genealogical-research journey comprises: determining a sequence for the set of discrete functions to achieve the genealogical-research target; and combining the set of discrete functions in the sequence.

CLAUSE 5: The computer-implemented method of any of clauses 1-4, further comprising: determining the advancing function from the set of discrete functions; generating a second genealogical-research journey that diverges from the genealogical-research journey and that includes a second set of discrete functions executable by the genealogical-data system; and reverting from the second genealogical-research journey back to the genealogical-research journey upon completing the second set of discrete functions by determining an additional advancing function from the set of discrete functions.

CLAUSE 6: The computer-implemented method of any of clauses 1-5, further comprising: detecting a journey metric indicating an inaction associated with executing the advancing function; and in response to detecting the journey metric, determining, utilizing a journey orchestration model, an alternative advancing function to progress the genealogical-research journey toward the genealogical-research target upon execution.

CLAUSE 7: The computer-implemented method of any of clauses 1-6, further comprising determining, utilizing a journey orchestration model, a skill set from a plurality of skill sets for executing the advancing function.

CLAUSE 8: The computer-implemented method of any of clauses 1-7, further comprising: receiving, from the client device, an interaction with a journey orchestration model, wherein the journey orchestration model can agentically access a plurality of skill sets; and in response to receiving the interaction with the journey orchestration model, providing, for display via the client device, a visualization associated with the advancing function to progress the genealogical-research journey toward the genealogical-research target upon execution utilizing a skill set of the plurality of skill sets.

CLAUSE 9: A system comprising: at least one processor; and at least one non-transitory computer-readable storage medium storing instructions that, when executed by the at least one processor, cause the system to: generate, for a user account within a genealogical-data system, a genealogical-research journey comprising a set of discrete functions executable by the genealogical-data system, the genealogical-research journey achieving a genealogical-research target upon execution of the set of discrete functions; determine, based on user-account data, a journey-progress indicator defining a subset of discrete functions from the set of discrete functions executable by the genealogical-data system during the genealogical-research journey; determine, based on the journey-progress indicator, an advancing function which progresses the genealogical-research journey toward the genealogical-research target upon execution; and provide a notification of the advancing function for display on a client device associated with the user account.

CLAUSE 10: The system of clause 9, further comprising instructions that, when executed by the at least one processor, cause the system to determine the user-account data based on at least one of the following: a level of engagement of the user account of the genealogical-data system with the genealogical-data system; user-account content associated with the user account comprised within a user-account database; and a user skill set associated with the user account.

CLAUSE 11: The system of any of clauses 9-10, further comprising instructions that, when executed by the at least one processor, cause the system to: generate a plurality of candidate genealogical-research targets based on the user-account data; and determine the genealogical-research target in response to receiving, from the client device, a user interaction corresponding to a particular genealogical-research target of the plurality of candidate genealogical-research targets.

CLAUSE 12: The system of any of clauses 9-11, further comprising instructions that, when executed by the at least one processor, cause the system to generate the genealogical-research journey by: determining a sequence for the set of discrete functions to achieve the genealogical-research target; and combining the set of discrete functions in the sequence.

CLAUSE 13: The system of any of clauses 9-12, further comprising instructions that, when executed by the at least one processor, cause the system to: determine the advancing function from the set of discrete functions; generate a second genealogical-research journey that diverges from the genealogical-research journey and that includes a second set of discrete functions executable by the genealogical-data system; and revert from the second genealogical-research journey back to the genealogical-research journey upon completing the second set of discrete functions by determining an additional advancing function from the set of discrete functions.

CLAUSE 14: The system of any of clauses 9-13, further comprising instructions that, when executed by the at least one processor, cause the system to: detect a journey metric indicating an inaction associated with executing the advancing function; and in response to detecting the journey metric, determine, utilizing a journey orchestration model, an alternative advancing function to progress the genealogical-research journey toward the genealogical-research target upon execution.

CLAUSE 15: The system of any of clauses 9-14, further comprising instructions that, when executed by the at least one processor, cause the system to: receive, from the client device, an interaction with a journey orchestration model, wherein the journey orchestration model can agentically access a plurality of skill sets; and in response to receiving the interaction with the journey orchestration model, provide, for display via the client device, a visualization associated with the advancing function to progress the genealogical-research journey toward the genealogical-research target upon execution utilizing a skill set of the plurality of skill sets.

CLAUSE 16: A non-transitory computer-readable medium storing instructions that, when executed by at least one processor, cause a computing device to: generate, for a user account within a genealogical-data system, a genealogical-research journey comprising a set of discrete functions executable by the genealogical-data system, the genealogical-research journey achieving a genealogical-research target upon execution of the set of discrete functions; determine, based on user-account data, a journey-progress indicator defining a subset of discrete functions from the set of discrete functions executable by the genealogical-data system during the genealogical-research journey; determine, based on the journey-progress indicator, an advancing function which progresses the genealogical-research journey toward the genealogical-research target upon execution; and provide a notification of the advancing function for display on a client device associated with the user account.

CLAUSE 17: The non-transitory computer-readable medium of clause 16, further comprising instructions that, when executed by the at least one processor, cause the computing device to: generate a plurality of candidate genealogical-research targets based on the user-account data; and determine the genealogical-research target in response to receiving, from the client device, a user interaction corresponding to a particular genealogical-research target of the plurality of candidate genealogical-research targets.

CLAUSE 18: The non-transitory computer-readable medium of any of clauses 16-17, further comprising instructions that, when executed by the at least one processor, cause the computing device to: determine the advancing function from the set of discrete functions; generate a second genealogical-research journey that diverges from the genealogical-research journey and that includes a second set of discrete functions executable by the genealogical-data system; and revert from the second genealogical-research journey back to the genealogical-research journey upon completing the second set of discrete functions by determining an additional advancing function from the set of discrete functions.

CLAUSE 19: The non-transitory computer-readable medium of any of clauses 16-18, further comprising instructions that, when executed by the at least one processor, cause the computing device to: detect a journey metric indicating an inaction associated with executing the advancing function; and in response to detecting the journey metric, determine, utilizing a journey orchestration model, an alternative advancing function to progress the genealogical-research journey toward the genealogical-research target upon execution.

CLAUSE 20: The non-transitory computer-readable medium of any of clauses 16-19, further comprising instructions that, when executed by the at least one processor, cause the computing device to: receive, from the client device, an interaction with a journey orchestration model, wherein the journey orchestration model can agentically access a plurality of skill sets; and in response to receiving the interaction with the journey orchestration model, provide, for display via the client device, a visualization associated with the advancing function to progress the genealogical-research journey toward the genealogical-research target upon execution utilizing a skill set of the plurality of skill sets.

It will be appreciated that while the genealogical-guidance system 100 embodiments have been described, the disclosure is not limited thereto. Rather, the disclosure extends to any suitable application of the disclosed embodiments in any suitable context, such as with regards to financial records, medical records, legal records, or other data types.

Embodiments of the present disclosure may comprise or utilize a special purpose or general-purpose computer including computer hardware, such as, for example, one or more processors and system memory, as discussed in greater detail below. Implementations within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, implementations of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some implementations, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multiprocessor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Implementations of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 10 illustrates a block diagram of exemplary computing device 1000 (e.g., the server device(s) 102 and/or the client device 108) that may be configured to perform one or more of the processes described above. One will appreciate that server device(s) 102 and/or the client device 108 may comprise one or more computing devices such as computing device 1000. As shown by FIG. 10, computing device 1000 can comprise processor 1002, memory 1004, storage device 1006, I/O interface 1008, and communication interface 1010, which may be communicatively coupled by way of communication infrastructure 1012. While an exemplary computing device 1000 is shown in FIG. 10, the components illustrated in FIG. 10 are not intended to be limiting. Additional or alternative components may be used in other implementations. Furthermore, in certain implementations, computing device 1000 can include fewer components than those shown in FIG. 10. Components of computing device 1000 shown in FIG. 10 will now be described in additional detail.

In particular implementations, processor 1002 includes hardware for executing instructions, such as those making up a computer program. As an example and not by way of limitation, to execute instructions, processor 1002 may retrieve (or fetch) the instructions from an internal register, an internal cache, memory 1004, or storage device 1006 and decode and execute them. In particular implementations, processor 1002 may include one or more internal caches for data, instructions, or addresses. As an example and not by way of limitation, processor 1002 may include one or more instruction caches, one or more data caches, and one or more translation lookaside buffers (TLBs). Instructions in the instruction caches may be copies of instructions in memory 1004 or storage device 1006.

Memory 1004 may be used for storing data, metadata, and programs for execution by the processor(s). Memory 1004 may include one or more of volatile and non-volatile memories, such as Random Access Memory (“RAM”), Read Only Memory (“ROM”), a solid state disk (“SSD”), Flash, Phase Change Memory (“PCM”), or other types of data storage. Memory 1004 may be internal or distributed memory.

Storage device 1006 includes storage for storing data or instructions. As an example and not by way of limitation, storage device 1006 can comprise a non-transitory storage medium described above. Storage device 1006 may include a hard disk drive (HDD), a floppy disk drive, flash memory, an optical disc, a magneto-optical disc, magnetic tape, or a Universal Serial Bus (USB) drive or a combination of two or more of these. Storage device 1006 may include removable or non-removable (or fixed) media, where appropriate. Storage device 1006 may be internal or external to computing device 1000. In particular implementations, storage device 1006 is non-volatile, solid-state memory. In other implementations, Storage device 1006 includes read-only memory (ROM). Where appropriate, this ROM may be mask programmed ROM, programmable ROM (PROM), erasable PROM (EPROM), electrically erasable PROM (EEPROM), electrically alterable ROM (EAROM), or flash memory or a combination of two or more of these.

I/O interface 1008 allows a user to provide input to, receive output from, and otherwise transfer data to and receive data from computing device 1000. I/O interface 1008 may include a mouse, a keypad or a keyboard, a touch screen, a camera, an optical scanner, network interface, modem, other known I/O devices or a combination of such I/O interfaces. I/O interface 1008 may include one or more devices for presenting output to a user, including, but not limited to, a graphics engine, a display (e.g., a display screen), one or more output drivers (e.g., display drivers), one or more audio speakers, and one or more audio drivers. In certain implementations, I/O interface 1008 is configured to provide graphical data to a display for presentation to a user. The graphical data may be representative of one or more graphical user interfaces and/or any other graphical content as may serve a particular implementation.

Communication interface 1010 can include hardware, software, or both. In any event, communication interface 1010 can provide one or more interfaces for communication (such as, for example, packet-based communication) between computing device 1000 and one or more other computing devices or networks. As an example and not by way of limitation, communication interface 1010 may include a network interface controller (NIC) or network adapter for communicating with an Ethernet or other wire-based network or a wireless NIC (WNIC) or wireless adapter for communicating with a wireless network, such as a WI-FI.

Additionally or alternatively, communication interface 1010 may facilitate communications with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, communication interface 1010 may facilitate communications with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination thereof.

Additionally, communication interface 1010 may facilitate communications various communication protocols. Examples of communication protocols that may be used include, but are not limited to, data transmission media, communications devices, Transmission Control Protocol (“TCP”), Internet Protocol (“IP”), File Transfer Protocol (“FTP”), Telnet, Hypertext Transfer Protocol (“HTTP”), Hypertext Transfer Protocol Secure (“HTTPS”), Session Initiation Protocol (“SIP”), Simple Object Access Protocol (“SOAP”), Extensible Mark-up Language (“XML”) and variations thereof, Simple Mail Transfer Protocol (“SMTP”), Real-Time Transport Protocol (“RTP”), User Datagram Protocol (“UDP”), Global System for Mobile Communications (“GSM”) technologies, Code Division Multiple Access (“CDMA”) technologies, Time Division Multiple Access (“TDMA”) technologies, Short Message Service (“SMS”), Multimedia Message Service (“MMS”), radio frequency (“RF”) signaling technologies, Long Term Evolution (“LTE”) technologies, wireless communication technologies, in-band and out-of-band signaling technologies, and other suitable communications networks and technologies.

Communication infrastructure 1012 may include hardware, software, or both that couples components of computing device 1000 to each other. As an example and not by way of limitation, communication infrastructure 1012 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination thereof.

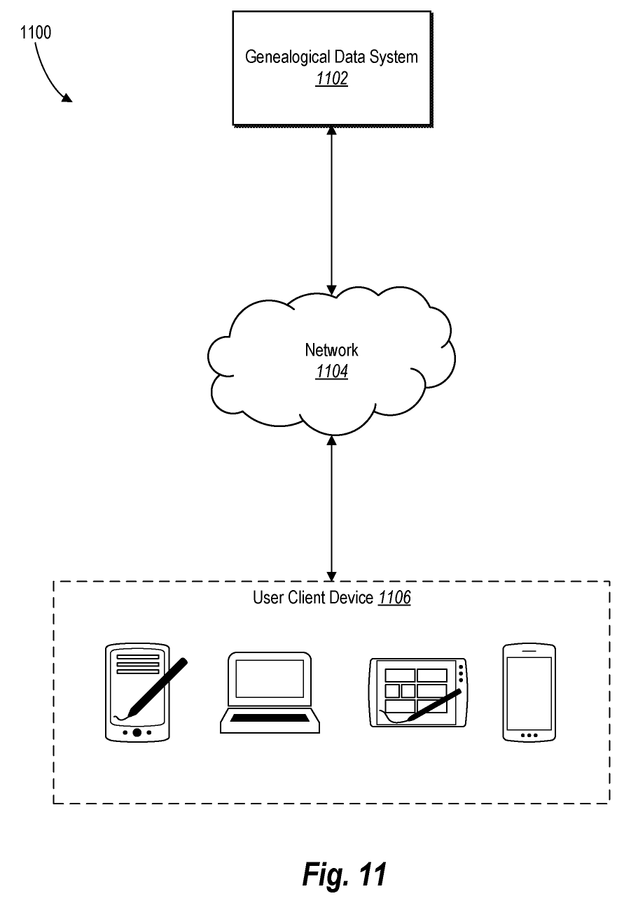

FIG. 11 is a schematic diagram illustrating environment 1100 within which one or more implementations of the genealogical-guidance system 100 can be implemented. For example, the genealogical-guidance system 100 may be part of a genealogical-data system 1102 (e.g., the genealogical-data system 104). The genealogical-data system 1102 may generate, store, manage, receive, and send digital content (such as genealogical content items). For example, genealogical-data system 1102 may send and receive digital content to and from client devices 1106 by way of network 1104. In particular, genealogical-data system 1102 can store and manage genealogical databases for various user accounts, historical records, genealogical records, and genealogy trees. In some embodiments, the genealogical-data system 1102 can manage the distribution and sharing of digital content between computing devices associated with user accounts. For instance, the genealogical-data system 1102 can facilitate a user account sharing a genealogical content item with another user account of genealogical-data system 1102.

In particular, the genealogical-data system 1102 can manage synchronizing digital content across multiple client devices 1106 associated with one or more user accounts. For example, a user may edit a digitized historical document or a node within a genealogy tree using client device 1106. The genealogical-data system 1102 can cause client device 1106 to send the edited genealogical content to the genealogical-data system 1102, whereupon the genealogical-data system 1102 synchronizes the genealogical content on one or more additional computing devices.

As shown, the client device 1106 may be a desktop computer, a laptop computer, a tablet computer, an augmented reality device, a virtual reality device, a personal digital assistant (PDA), an in- or out-of-car navigation system, a handheld device, a smart phone or other cellular or mobile phone, or a mobile gaming device, other mobile device, or other suitable computing devices. The client device 1106 may execute one or more client applications, such as a web browser (e.g., Microsoft Windows Internet Explorer, Mozilla Firefox, Apple Safari, Google Chrome, Opera, etc.) or a native or special-purpose client application (e.g., Ancestry: Family History & DNA for iPhone or iPad, Ancestry: Family History & DNA for Android, etc.), to access and view content over the network 1104.

The network 1104 may represent a network or collection of networks (such as the Internet, a corporate intranet, a virtual private network (VPN), a local area network (LAN), a wireless local area network (WLAN), a cellular network, a wide area network (WAN), a metropolitan area network (MAN), or a combination of two or more such networks) over which client devices 1106 may access genealogical-data system 1102.

In the foregoing specification, the present disclosure has been described with reference to specific exemplary implementations thereof. Various implementations and aspects of the present disclosure(s) are described with reference to details discussed herein, and the accompanying drawings illustrate the various implementations. The description above and drawings are illustrative of the disclosure and are not to be construed as limiting the disclosure. Numerous specific details are described to provide a thorough understanding of various implementations of the present disclosure.

The present disclosure may be embodied in other specific forms without departing from its spirit or essential characteristics. The described implementations are to be considered in all

respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

What is claimed is:

1. A computer-implemented method comprising:
generating, for a user account within a genealogical-data system, a genealogical-research journey comprising a set of discrete functions executable by the genealogical-data system, the genealogical-research journey achieving a genealogical-research target upon execution of the set of discrete functions;
determining, based on user-account data, a journey-progress indicator defining a subset of discrete functions from the set of discrete functions executable by the genealogical-data system during the genealogical-research journey;
determining, based on the journey-progress indicator, an advancing function which progresses the genealogical-research journey toward the genealogical-research target upon execution; and
providing a notification of the advancing function for display on a client device associated with the user account.

2. The computer-implemented method of claim 1, further comprising determining the user-account data based on at least one of the following:
a level of engagement of the user account of the genealogical-data system with the genealogical-data system;
user-account content associated with the user account comprised within a user-account database; and
a user skill set associated with the user account.

3. The computer-implemented method of claim 1, further comprising:
generating a plurality of candidate genealogical-research targets based on the user-account data; and
determining the genealogical-research target in response to receiving, from the client device, a user interaction corresponding to a particular genealogical-research target of the plurality of candidate genealogical-research targets.

4. The computer-implemented method of claim 1, wherein generating the genealogical-research journey comprises:
determining a sequence for the set of discrete functions to achieve the genealogical-research target; and
combining the set of discrete functions in the sequence.

5. The computer-implemented method of claim 1, further comprising:
determining the advancing function from the set of discrete functions;
generating a second genealogical-research journey that diverges from the genealogical-research journey and that includes a second set of discrete functions executable by the genealogical-data system; and
reverting from the second genealogical-research journey back to the genealogical-research journey upon completing the second set of discrete functions by determining an additional advancing function from the set of discrete functions.

6. The computer-implemented method of claim 1, further comprising:
detecting a journey metric indicating an inaction associated with executing the advancing function; and
in response to detecting the journey metric, determining, utilizing a journey orchestration model, an alternative advancing function to progress the genealogical-research journey toward the genealogical-research target upon execution.

7. The computer-implemented method of claim 1, further comprising determining, utilizing a journey orchestration model, a skill set from a plurality of skill sets for executing the advancing function.

8. The computer-implemented method of claim 1, further comprising:
receiving, from the client device, an interaction with a journey orchestration model, wherein the journey orchestration model can agentically access a plurality of skill sets; and
in response to receiving the interaction with the journey orchestration model, providing, for display via the client device, a visualization associated with the advancing function to progress the genealogical-research journey toward the genealogical-research target upon execution utilizing a skill set of the plurality of skill sets.

9. A system comprising:
at least one processor; and
at least one non-transitory computer-readable storage medium storing instructions that, when executed by the at least one processor, cause the system to:
generate, for a user account within a genealogical-data system, a genealogical-research journey comprising a set of discrete functions executable by the genealogical-data system, the genealogical-research journey achieving a genealogical-research target upon execution of the set of discrete functions;
determine, based on user-account data, a journey-progress indicator defining a subset of discrete functions from the set of discrete functions executable by the genealogical-data system during the genealogical-research journey;
determine, based on the journey-progress indicator, an advancing function which progresses the genealogical-research journey toward the genealogical-research target upon execution; and
provide a notification of the advancing function for display on a client device associated with the user account.

10. The system of claim 9, further comprising instructions that, when executed by the at least one processor, cause the system to determine the user-account data based on at least one of the following:
a level of engagement of the user account of the genealogical-data system with the genealogical-data system;
user-account content associated with the user account comprised within a user-account database; and
a user skill set associated with the user account.

11. The system of claim 9, further comprising instructions that, when executed by the at least one processor, cause the system to:
generate a plurality of candidate genealogical-research targets based on the user-account data; and
determine the genealogical-research target in response to receiving, from the client device, a user interaction corresponding to a particular genealogical-research target of the plurality of candidate genealogical-research targets.

12. The system of claim 9, further comprising instructions that, when executed by the at least one processor, cause the system to generate the genealogical-research journey by:
determining a sequence for the set of discrete functions to achieve the genealogical-research target; and
combining the set of discrete functions in the sequence.

13. The system of claim 9, further comprising instructions that, when executed by the at least one processor, cause the system to:
determine the advancing function from the set of discrete functions;
generate a second genealogical-research journey that diverges from the genealogical-research journey and that includes a second set of discrete functions executable by the genealogical-data system; and
revert from the second genealogical-research journey back to the genealogical-research journey upon completing the second set of discrete functions by determining an additional advancing function from the set of discrete functions.

14. The system of claim 9, further comprising instructions that, when executed by the at least one processor, cause the system to:
detect a journey metric indicating an inaction associated with executing the advancing function; and
in response to detecting the journey metric, determine, utilizing a journey orchestration model, an alternative advancing function to progress the genealogical-research journey toward the genealogical-research target upon execution.

15. The system of claim 9, further comprising instructions that, when executed by the at least one processor, cause the system to:
receive, from the client device, an interaction with a journey orchestration model, wherein the journey orchestration model can agentically access a plurality of skill sets; and
in response to receiving the interaction with the journey orchestration model, provide, for display via the client device, a visualization associated with the advancing function to progress the genealogical-research journey toward the genealogical-research target upon execution utilizing a skill set of the plurality of skill sets.

16. A non-transitory computer-readable medium storing instructions that, when executed by at least one processor, cause a computing device to:
generate, for a user account within a genealogical-data system, a genealogical-research journey comprising a set of discrete functions executable by the genealogical-data system, the genealogical-research journey achieving a genealogical-research target upon execution of the set of discrete functions;
determine, based on user-account data, a journey-progress indicator defining a subset of discrete functions from the set of discrete functions executable by the genealogical-data system during the genealogical-research journey;
determine, based on the journey-progress indicator, an advancing function which progresses the genealogical-research journey toward the genealogical-research target upon execution; and
provide a notification of the advancing function for display on a client device associated with the user account.

17. The non-transitory computer-readable medium of claim 16, further comprising instructions that, when executed by the at least one processor, cause the computing device to:
generate a plurality of candidate genealogical-research targets based on the user-account data; and
determine the genealogical-research target in response to receiving, from the client device, a user interaction corresponding to a particular genealogical-research target of the plurality of candidate genealogical-research targets.

18. The non-transitory computer-readable medium of claim 16, further comprising instructions that, when executed by the at least one processor, cause the computing device to:
determine the advancing function from the set of discrete functions;
generate a second genealogical-research journey that diverges from the genealogical-research journey and that includes a second set of discrete functions executable by the genealogical-data system; and
revert from the second genealogical-research journey back to the genealogical-research journey upon completing the second set of discrete functions by determining an additional advancing function from the set of discrete functions.

19. The non-transitory computer-readable medium of claim 16, further comprising instructions that, when executed by the at least one processor, cause the computing device to:
detect a journey metric indicating an inaction associated with executing the advancing function; and
in response to detecting the journey metric, determine, utilizing a journey orchestration model, an alternative advancing function to progress the genealogical-research journey toward the genealogical-research target upon execution.

20. The non-transitory computer-readable medium of claim 16, further comprising instructions that, when executed by the at least one processor, cause the computing device to:
receive, from the client device, an interaction with a journey orchestration model, wherein the journey orchestration model can agentically access a plurality of skill sets; and
in response to receiving the interaction with the journey orchestration model, provide, for display via the client device, a visualization associated with the advancing function to progress the genealogical-research journey toward the genealogical-research target upon execution utilizing a skill set of the plurality of skill sets.
